(19) United States
(12) Patent Application Publication
(10) Pub. No.: US 2024/0369522 A1
(43) Pub. Date: Nov. 7, 2024

(54) METHOD FOR ESTIMATING THE BIOMASS OF HAIRY ROOTS IN A MULTI-PHASIC CULTURE

(71) Applicant: SAMABRIVA, Amiens (FR)

(72) Inventors: Florian CARDON, Amiens (FR); Camille LEMASSON, Amiens (FR); Marina GUILLET, Amiens (FR)

(73) Assignee: SAMABRIVA, Amiens (FR)

(21) Appl. No.: 18/573,003

(22) PCT Filed: Jun. 22, 2022
(86) PCT No.: PCT/EP2022/067056

§ 371 Date: Dec. 21, 2023

(30) Foreign Application Priority Data
Jun. 22, 2021 (EP) 21305854.8

(51) Int. Cl. G01N 33/00; A01G 7/00; A01H 4/00

(52) U.S. Cl. CPC G01N 33/0098; A01G 7/00; A01H 4/00

(57) ABSTRACT

A method for estimating the hairy root biomass concentration X₍ₜ₎ of a multi-phasic culture of hairy roots in a culture medium, the culture medium including at least one source of carbon being at least one sugar, and at least one source of nitrogen. The biomass yield surprisingly correlates linearly with the consumed concentration of nitrogen and/or sugars over the entire time course of the multi-phasic culture of hairy roots. The method for estimating the hairy root biomass concentration X₍ₜ₎ according to the invention is also a useful tool to estimate the amount of one or more molecule(s) of interest synthesized by the hairy roots.

### FIELD OF INVENTION

The present invention relates to a method for estimating the biomass of hairy roots in a multi-phasic culture of hairy roots in a culture medium.

### BACKGROUND OF INVENTION

Hairy roots have been widely studied and used for the production of specialized/secondary metabolites of industrial and pharmaceutical interest. Since the 1990s, the production of recombinant proteins has been considered as another promising application of hairy root cultures. Indeed, this plant-based technology offers relevant and advantageous differences as compared to classical expression systems (such as, e.g., Chinese hamster ovary (CHO) cells, human cell lines, bacteria).

As a matter of fact, the selected producing hairy root clones are easily grown in tailor-made optimal conditions in a simple culture medium with a better safety profile than media containing human- or animal-derived constituents. The hairy root-based expression system allows for the production of specialized/secondary metabolites, as well as recombinant proteins, in an axenic environment in large bioreactors, such as 350 L bioreactors. The hairy root-based expression system presents similarities with the expression systems used to produce recombinant proteins from mammalian cell lines, among which the fact that the whole process is maintained under sterile conditions in a confined bioreactor.

To be used for the production of therapeutic compounds, the development of hairy root-based expression systems compatible with industrial and good manufacturing practices (GMP) is essential. Unlike most of the other plant-based expression systems, the hairy roots are not cultured in open field/in the wild as described in Table 1 of the Annex 2 of the GMP EU guidelines “Manufacture of Biological active substances and Medicinal Products for Human Use” and in the HMPC (Committee on Herbal Medicinal Products) guidelines on Good Agriculture and Collection Practice.

Thus, hairy root platforms offer the advantage that the whole process is maintained in a closed system providing a sterile environment, from the establishment of the Master Root Bank (equivalent of the Master Cell Bank) up to the filling, including the growing and harvesting steps. It would thus be conceivable for the whole process to be developed according to the GMP requirements since the system is similar to the system described for recombinant proteins manufactured from cell banks. This particularity of hairy root platforms would largely facilitate the implementation of the GMP quality system.

Additionally, implementing GMP and quality control requires the ability to be able to estimate hairy root biomass growth in real-time during the whole process. Indeed, hairy root biomass growth is a good indicator of the quality of a hairy root culture, i.e., of the quality of a process of production in a hairy root-based expression system. For example, a reduced hairy root biomass growth reflects a suboptimal hairy root culture, i.e., a suboptimal process of production in a hairy root-based expression system. Of note, the hairy root biomass may not accessible for direct measure while the production process is on-going in the closed system required for ensuring a sterile environment. Therefore, biomarkers are needed to estimate hairy root biomass growth in real-time during an on-going hairy root culture, i.e., during an on-going process of production in a hairy root-based expression system.

However, identification of biomarkers to follow or monitor biomass growth over the time course of the different phases of a hairy root multi-phasic culture, comprising for example a phase of hairy root growth and a phase of production of compounds of interest, is rendered difficult by the changes in kinetic and/or metabolic parameters during the different phases.

Therefore, there is a need to provide the state of the art with biomarkers that allow to estimate biomass growth in real-time, at any moment during the time course of an on-going multi-phasic hairy root culture, in particular a multi-phasic hairy root culture intended to produce biomass and/or compounds of interest.

### SUMMARY

The present invention relates to a method for estimating the hairy root biomass concentration X₍ₜ₎ of a multi-phasic culture of hairy roots in a culture medium, the culture medium comprising at least one source of carbon being at least one sugar, and one source of nitrogen, the method comprising:
a) measuring, at time t₀, the concentration C₀ in the culture medium of at least one compound selected from the group consisting of at least one sugar and at least one nitrogen source;
b) measuring, at a time t of any one of the culture phases, the concentration C₍ₜ₎ in the culture medium of the at least one compound;
c) calculating the differential concentration C₀-C₍ₜ₎ of the at least one compound;
d) estimating the hairy root biomass concentration X₍ₜ₎ at the time t of any one of the culture phases by means of the following equation:

$$\\ {X}_{( t )} = [ {Y}_{X / i} \times ( {C}_{0} - {C}_{( t )} ) ] + {X}_{0} ,$$

wherein:
X₀ is the hairy root biomass concentration at time t₀, and
Y_{X/i} is the apparent biomass yield coefficient empirically pre-determined by means of the following equation:

$$\\ {Y}_{X / i} = \frac{{X}_{1} - {X}_{0}}{{C}_{0} - {C}_{1}}$$

wherein X₁ is the hairy root biomass concentration at a predetermined time t₁;
C₁ is the concentration of the at least one compound at a predetermined time t₁;
X₀ is the hairy root biomass concentration at time t₀; C₀ is the concentration of the at least one compound at time t₀; and i is S for sugar(s) or N for nitrogen.

In some embodiments, the at least one sugar is selected from the group comprising or consisting of glucose, fructose, sucrose, and any combinations thereof, and preferably consists of a combination of glucose, fructose and sucrose.

In some embodiments, each phase of the multi-phasic culture is characterized by its own biomass growth rate.

In some embodiments, the multi-phasic culture is a bi-phasic culture. In some embodiments, the multi-phasic culture, in particular the bi-phasic culture, comprises:
a) a first phase of culturing hairy roots dedicated to biomass growth; and
b) one or more further phase(s) of culturing hairy roots dedicated to the production of one or more molecule(s) of interest.

In some embodiments, the multi-phasic culture of hairy roots is aimed at producing fresh biomass and/or one or more molecule(s) of interest. In some embodiments, the one or more molecule(s) of interest is/are selected from the group comprising or consisting of recombinant proteins, metabolites, non-peptidic hormones, structured associations of recombinant proteins, virus-like particles and viruses. In some embodiments, the recombinant protein is selected from the group comprising or consisting of allergens; vaccines; viral proteins; enzymes; enzyme inhibitors; antibodies; antibody fragments; antigens, toxins; anti-microbial peptides; peptidic hormones; growth factors; blood proteins, in particular albumin, coagulation factors, transferrin; receptors and/or signaling proteins; protein components of biomedical standards; protein components of cell culture media; fusion and/or tagged proteins; cysteine (disulfide bridges)-rich peptides and proteins; and plant proteins, in particular lectins, papain. In some embodiments, the metabolite is selected from the group comprising or consisting of polyphenols; alkaloids; cannabinoids; terpenoids and steroids; flavonoids; and tannins.

In some embodiments, the hairy root is selected from the group of families comprising or consisting of the Brassicaceae family; the Solanaceae family; the Cannabaceae family; the Caryophyllaceae family; the Saponaria family; and the Vitaceae family. In some embodiments, the hairy root is selected from the group of species comprising or consisting of Brassica rapa rapa, Brassica napus, Salvia Milthiorrhiza, Panax Ginseng, Armoracia rusticana, Trigonella foenumgraceum, Lippia dulcis, Lithospermum erythrorhizon, Ophiorrhiza pumila, and Echinacea purpurea, Echinacea Angustifolia, Puerariaphaseoloides, Harpagophytum Procumbens, Morinda Citrifolia, Hypericum Perforatum, Derris trifolia, Salvia miltiorrhiza, Salvia prevalzkii, Echinacea pallida, Cistanche tubulosa, Glycyrrhiza glabra, Sophora flavescens, Rhodiola Rosea, Polygonum cuspidatum, Fallopia multiflora, Lepidium peruvianum, Whitania Somnifera, Astragalus Membranaceous, Berberis Vulgaris, Sanguinaria canadensis, Eleutherococcus Senticosus, Cannabis sativa, Hydrastis Canadensis, Arctium Majus, Piper methysticium, Pueraria lobata, Glycyrrhiza uralensis, Ptychopetalum olacoides, Dioscorea Vollosa, Yucca shidigera, Panax quinquifolium, Azadirachta indica, Catharanthus trichophyllus, Calystegia sepium, Atropa belladonna, Hyoscyamus muticus, Artemisia annua, Datura stramonium, Arabidopsis thaliana, Stizolobium, Hassjoo, Ipomea aquatica, Perilla fruitescnens, Catharanthus roseus, Taxus brevifolia, Gloriosa Superba, Saponaria officinalis, Solanum tuberosum, Nicotiana tabacum, Nicotiana benthamiana, Cannabis sativa, Vitis vinifera, Duboisia leichhardtii, Ducoisia myoporoides and Cinchosa Pubescens.

In some embodiments, the one or more further phase(s) of culturing the hairy roots is/are performed in the presence of a chemical and/or a physical and/or biological inductor of the production of the one or more molecule(s) of interest.

In some embodiments, when the at least one compound is at least one sugar, the biomass yield Y_{X/S} ranges from about 0.25 to about 2.50. In some embodiments, when the at least one compound is a nitrogen source, the biomass yield Y_{X/N} ranges from about 5 to about 40.

In some embodiments, the multi-phasic culture is performed in a bioreactor, preferably in a volume of culture medium of at least about 20 L, more preferably of at least about 350 L, more preferably at about 500 L.

### Definitions

In the present invention, the following terms have the following meanings:

“About” preceding a figure encompasses plus or minus 10%, or less, of the value of said figure. It is to be understood that the value to which the term “about” refers to is itself also specifically, and preferably, disclosed.

“Comprise” is intended to mean “contain”, “encompass” and “include”. In some embodiments, the term “comprise” also encompasses the term “consist of”.

“Hairy roots” refers to root emergences which appear after the infection of a plant by Rhizobium rhizogenes (previously referred to as Agrobacterium rhizogenes) bacteria or by Rhizobium radiobacter (also known as Agrobacterium Tumefaciens) bacteria harboring rol genes for example.

“Hairy roots-based expression system” (also sometimes referred to as “hairy root system” or “hairy root expression system”) refers to a culture of hairy roots, either previously engineered (for example genetically modified) or not, which purpose is to synthesize a compound or molecule of interest (such as a metabolite or a recombinant protein), allowing in fine the hairy roots to produce, and optionally secrete, said compound.

“Biomass”, as used herein, refers to the weight of plant dry or fresh matter, in particular of hairy roots dry or fresh matter, at a given time of a plant culture, in particular a culture of hairy roots.

“Biomass concentration”, as used herein, refers to the ratio of the quantity of biomass (expressed as weight) over the volume of culture. In practice, the biomass concentration is expressed as g·L⁻¹.

“Dry biomass”, as used herein, refers to the weight of plant, in particular of hairy roots, upon removal of the water, e.g., by water evaporation.

“Biomass yield” refers to the ratio of the amount (or quantity) of biomass produced over the amount (or quantity) of nutrient consumed either measured directly (g biomass/g nutrient) or estimated indirectly through the measure of the medium conductivity (g biomass/mS). In some embodiments, “biomass yield” and “apparent biomass yield” are intended to substitute for one another.

“Biomass growth” may refer to the process of culturing a plant, in particular hairy roots, in order to increase its weight, as measured for example by its weight of dry or fresh matter. “Biomass growth” may also refer to the production or increase of biomass, for example of hairy root biomass, over the course of a culture, for example of a hairy root culture.

“Multi-phasic culture” refers to a culture which possesses at least 2 distinct phases, such as, e.g., a phase dedicated to the growth of biomass itself (referred to herein as “first phase”), and (i) one or more phase(s) dedicated to the production of one or more compound(s) or molecule(s) of interest (referred to herein as “further phase(s)”), including successive phases dedicated to the production of one or more compound(s) or molecule(s) of interest, and/or (ii) one or more phase(s) of maintenance of the biomass without growth. Illustratively, the one or more further phase(s) dedicated to the production of the one or more compound(s) or molecule(s) of interest may or may not promote the growth of biomass.

“Growth rate” may refer to the speed at which the number of living organisms, in particular hairy roots, within a population, increases during the time course of a culture of said living organisms, in particular hairy roots. “Growth rate” may also preferably refer to the increase of biomass over the duration of a culture, in particular a culture of hairy roots.

“Culture medium” refers to a solid or liquid medium containing all the required nutrients for a culture, in particular for a culture of hairy roots.

“Nutrients” refers to the compounds that are present in a culture medium and consumed, in particular by hairy roots, in order to produce biomass and/or to produce one or more compound(s) or molecule(s) of interest. In some embodiments, the nutrients include sugars, such as, e.g., sucrose; sulfate; phosphate; nitrate; ammonium; potassium; and the like.

“Biomarker” refers to a characteristic or parameter that may be objectively measured, assessed and/or evaluated as an indicator of normal biological processes, or biological responses to the environment's changes. Illustratively, such characteristic or parameter may be the consumption of a specific nutrient by a living organism, for example hairy roots, that is cultured in a suitable culture medium.

“Rhizocal” or “rhizocallus” refers to a conic-shaped structure connected to roots, also termed lateral root emergence, which develops alongside of roots in a solidarized way. In practice, the “rhizocals” or “rhizocalli” may be induced in a culture of roots by the addition of one or more agent(s) that promote(s) the induction of rhizocals in the culture medium, such as, e.g., the hormones called auxins or synthetic auxins such as 2,4-dichlorophenoxyacetic acid (2,4-D). The presence of “rhizocals” in a culture of hairy roots is often associated with a better yield in the production of a compound or molecule of interest by the hairy roots.

“Rhizocal induction” refers to a phase of culture of roots, in particular hairy roots, wherein the culture medium is supplemented with an agent, in particular a hormone, more specifically an auxin, such as, e.g., 2,4-D, which is capable of promoting the induction of rhizocals. The rhizocals enable an increase of the ability of the roots to produce and optionally secrete one or more compound(s) or molecule(s) of interest.

“Metabolites” refers to small molecules i.e., intermediate or final products of metabolic reactions, such as, e.g., polyphenols, alkaloids, cannabinoids, terpenoids, steroids, flavonoids and tannins. Valuable metabolites, i.e., metabolites of interest or its precursors, may be naturally synthesized and secreted by roots, in particular by hairy roots. Alternatively, the synthesis and secretion of valuable metabolites may be artificially induced in roots, in particular in hairy roots (for example by adding an inductor or elicitor to the culture medium, by exposing the roots to a stress and/or by genetically modifying the metabolic pathway).

“Recombinant protein” refers to a protein encoded by a DNA nucleic acid that has been cloned in a vector system that supports expression of the protein, including the transcription into a messenger RNA (mRNA) and translation of said messenger RNA into the protein. The expression “recombinant protein” or “protein of interest” are used interchangeably.

“to” as used herein refers to a time preceding the start of a multi-phasic culture of hairy roots. In some embodiments, “t₀” refers to the start of a multi-phasic culture of hairy roots. Accordingly, the concentration C₀ in the culture medium of a compound at time to is the concentration of said compound in the culture medium before the start of a multi-phasic culture of hairy roots or at the start of a multi-phasic culture of hairy roots. In other words, C₀ is the initial concentration of the compound in the culture medium. Similarly, the hairy root biomass concentration X₀ at time to is the hairy root biomass concentration in the culture medium before the start of a multi-phasic culture of hairy roots or at the start of a multi-phasic culture of hairy roots. In other words, X₀ is the initial hairy root biomass concentration in the culture medium.

### DETAILED DESCRIPTION

Because the evolution towards large liquid hairy root cultures in bioreactors to produce recombinant proteins or metabolites for industrial purpose is essential, an in-depth characterization of hairy root cultures was performed by the inventors in both small and large scale in order to identify critical parameters for biomass production. The inventors specifically focused on biomass in order to characterize cultures of transgenic and non-transgenic hairy roots of several species among which Brassica rapa rapa, Cannabis sativa and Brassica napus, which may produce recombinant proteins or metabolites. Moreover, identification of biomarkers to follow or monitor biomass growth overtime, essential to pilot a robust process, are presented in the example section below. This characterization has been done first in small-scale cultures by following the kinetics of nutrients consumption and production of biomass, to be finally compared with cultures in large-scale bioreactors. The inventors have thus shown that it is possible to correctly estimate the hairy root biomass concentration of an on-going hairy root culture, in particular of a multi-phasic hairy root culture. The real-time estimation of the hairy root biomass concentration at any time during a multi-phasic hairy root culture is particularly relevant to monitor said multi-phasic hairy root culture. For example, the real-time estimation of the hairy root biomass concentration at any time during a multi-phasic hairy root culture may serve as quality control. It may also be particularly helpful to make any adjustment or take any corrective step that may be required to ensure optimal hairy root biomass growth, and thus optimal culture quality. Of note, estimating the hairy root biomass concentration of an on-going hairy root culture may also be a useful tool to estimate the amount or quantity of one or more molecule(s) of interest produced, and optionally secreted, by the hairy roots.

The present invention relates to a method for estimating the hairy root biomass concentration X₍ₜ₎ of a multi-phasic culture of hairy roots in a culture medium, the culture medium comprising at least one source of carbon being at least one sugar, and at least one source of nitrogen, the method comprising:
a) measuring, at time t₀, the concentration C₀ in the culture medium of at least one compound selected from the group comprising or consisting of at least one sugar and at least one nitrogen source;
b) measuring, at a time t of any one of the culture phases, the concentration C₍ₜ₎ in the culture medium of the at least one compound;
c) calculating the differential concentration C₀-C₍ₜ₎ of the at least one compound;
d) estimating, preferably estimating in real-time, the hairy root biomass concentration X₍ₜ₎ at the time t of any one of the culture phases by means of the following equation:

$$\\ {X}_{( t )} = [ {Y}_{X / i} \times ( {C}_{0} - {C}_{( t )} ) ] + {X}_{0} ,$$

wherein:
X₀ is the hairy root biomass concentration at time t₀, and
Y_{X/i} is the apparent biomass yield coefficient (also referred to as biomass yield) empirically pre-determined by means of the following equation:

$$\\ {Y}_{X / i} = \frac{{X}_{1} - {X}_{0}}{{C}_{0} - {C}_{1}}$$

wherein X₁ is the hairy root biomass concentration at a predetermined time t₁;
C₁ is the concentration of the at least one compound at a predetermined time t₁;
X₀ is the hairy root biomass concentration at time t₀; C₀ is the concentration of the at least one compound at time t₀; and i is S for sugar(s) or N for nitrogen (in other words, i is S when the at least one compound is at least one sugar, and i is N when the at least one compound is at least one nitrogen source).

The present invention also relates to a method for estimating in real-time the hairy root biomass concentration X(t) of a multi-phasic culture of hairy roots in a culture medium, the culture medium comprising at least one source of carbon being at least one sugar, and at least one source of nitrogen, the method comprising steps a) to d) as described above.

In some embodiments, the multi-phasic culture of hairy roots is thus an on-going or existing multi-phasic culture of hairy roots, as opposed to a theoretical or modelized multi-phasic culture of hairy roots.

The present invention further relates to a method for controlling the quality, or implementing the quality control, of a multi-phasic culture of hairy roots in a culture medium comprising at least one source of carbon being at least one sugar, and at least one source of nitrogen, the method comprising steps a) to d) as described above, thereby controlling the quality of the multi-phasic culture of hairy roots, or implementing the quality control of the multi-phasic culture of hairy roots.

In some embodiments, steps b) to d) of the methods described herein can be performed several times over the time course of the multi-phasic culture of hairy roots, for example at least twice, at least three times, at least four times or at least five times. In some embodiments, steps b) to d) of the methods described herein can be performed at regular intervals over the time course of the multi-phasic culture of hairy roots, for example every 3, 4, 5, 6, 7, 8, 9, or 10 days. Thus, in some embodiments, the methods described herein comprise estimating, preferably estimating in real-time, the hairy root biomass concentration X₍ₜ₎ of the multi-phasic hairy root culture several times over the time course of the multi-phasic culture of hairy roots, for example at least twice, at least three times, at least four times or at least five times. In some embodiments, the methods described herein comprise estimating, preferably estimating in real-time, the hairy root biomass concentration X(t) of the multi-phasic hairy root culture at least twice over the time course of the multi-phasic culture of hairy roots. In some embodiments, the methods described herein comprise estimating, preferably estimating in real-time, the hairy root biomass concentration X(t) of the multi-phasic hairy root culture at regular intervals over the time course of the multi-phasic culture of hairy roots, for example every 3, 4, 5, 6, 7, 8, 9, or 10 days.

Accordingly, the present invention further relates to a method for monitoring a multi-phasic culture of hairy roots in a culture medium comprising at least one source of carbon being at least one sugar, and at least one source of nitrogen, the method comprising steps a) to d) as described above, wherein steps b) to d) are performed at least twice, so that the hairy root biomass concentration X₍ₜ₎ is estimated at least twice over the time course of the multi-phasic culture of hairy roots, thereby monitoring the multi-phasic culture of hairy roots.

In some embodiments, the hairy root biomass concentration X₍ₜ₎ estimated at step d) allows to detect an anomaly with the multi-phasic hairy root culture, such as a suboptimal biomass growth rate (μ) or the occurrence of a contamination by a microorganism. In some embodiments, estimating the hairy root biomass concentration X₍ₜ₎ several times over the time course of the multi-phasic culture of hairy roots, for example at least twice, allows to detect an anomaly with the multi-phasic hairy root culture, such as a suboptimal biomass growth rate (μ) or the occurrence of a contamination by a microorganism.

Thus, the present invention also relates to a method for controlling or maintaining the quality of a multi-phasic culture of hairy roots in a culture medium comprising at least one source of carbon being at least one sugar, and at least one source of nitrogen, the method comprising steps a) to d) as described above, thereby detecting an anomaly with the multi-phasic hairy root culture.

In some embodiments, a hairy root biomass concentration X₍ₜ₎ estimated at step d) which is substantially identical or similar to a reference value indicates the presence of an anomaly with the multi-phasic hairy root culture, for example indicates a suboptimal biomass growth rate (μ). In some embodiments, the expression “substantially identical or similar” is intended to mean that the hairy root biomass concentration X₍ₜ₎ is less than about 20%, 19%, 18%, 17%, 16%, 15%, 14%, 13%, 12%, 11%, 10%, 9%, 8%, 7%, 6%, 5%, 4%, 3%, 2%, or 1% higher than the reference value.

In some embodiments, the reference value is the hairy root biomass concentration X₍₀₎ at time to. Thus, in some embodiments, a hairy root biomass concentration X₍ₜ₎ estimated at step d) which is substantially identical (or similar) to the hairy root biomass concentration X₍₀₎ at time to indicates the presence of an anomaly with the multi-phasic hairy root culture, for example indicates a suboptimal biomass growth rate (μ), in particular when time t is at least about 3 days, more preferably at least about 4, 5, 6, 7, 8, 9, or 10 days of the culture of hairy roots, i.e., since the start of the culture of hairy roots.

As indicated above, steps b) to d) of the methods described herein can be performed several times over the time course of the multi-phasic culture of hairy roots, and the hairy root biomass concentration X₍ₜ₎ can thus be estimated several times over the time course of the multi-phasic culture of hairy roots. Accordingly, in some embodiments, the reference value is a prior hairy root biomass concentration X₍ₜ₎ (e.g., a first hairy root biomass concentration X₍ₜ₎) estimated for the on-going multi-phasic hairy root culture. Thus, in some embodiments, an ulterior hairy root biomass concentration X₍ₜ₎ (e.g., a second hairy root biomass concentration X₍ₜ₎) which is substantially identical (or similar) to a prior hairy root biomass concentration X₍ₜ₎ (e.g., a first hairy root biomass concentration X₍ₜ₎ indicates the presence of an anomaly with the multi-phasic hairy root culture, for example indicates a suboptimal biomass growth rate (μ), in particular when the ulterior hairy root biomass concentration X₍ₜ₎ is estimated at least about 3 days, more preferably at least about 4, 5, 6, 7, 8, 9, or 10 days after the estimation of the prior hairy root biomass concentration X₍ₜ₎.

In some embodiments, a hairy root biomass concentration X₍ₜ₎ estimated at step d) which is substantially different to a reference value indicates the presence of an anomaly with the multi-phasic hairy root culture, for example indicates a suboptimal biomass growth rate (μ). In some embodiments, the expression “substantially different” is intended to mean that the hairy root biomass concentration X₍ₜ₎ is more than about 1%, 2%, 3%, 4%, 5%, 6%, 7%, 8%, 9%, 10%, 11%, 12%, 13%, 14%, 15%, 16%, 17%, 18%, 19%, or 20% lower than the reference value. In some embodiments, the reference value is a predetermined reference value. For example, a predetermined reference value may be empirically derived from a multi-phasic hairy root culture conducted in similar conditions (for example, using the same species of hairy roots, and/or the same culture medium).

In some embodiments, the methods as described above further comprises a step e) of modifying or adapting or adjusting the conditions of the multi-phasic culture of hairy roots. In some embodiments, step e) of modifying or adapting or adjusting the conditions of the multi-phasic culture of hairy roots allows to correct the anomaly with the multi-phasic culture of hairy roots detected by estimating the hairy root biomass concentration X₍ₜ₎ at step d) as indicated above.

Thus, the present invention also relates to a method comprising:
a) measuring, at time t₀, the concentration C₀ in the culture medium of at least one compound selected from the group comprising or consisting of at least one sugar and at least one nitrogen source;
b) measuring, at a time t of any one of the culture phases, the concentration C₍ₜ₎ in the culture medium of the at least one compound;
c) calculating the differential concentration C₀-C₍ₜ₎ of the at least one compound;
d) estimating, preferably estimating in real-time, the hairy root biomass concentration X₍ₜ₎ at the time t of any one of the culture phases by means of the following equation:

$$\\ {X}_{( t )} = [ {Y}_{X / i} \times ( {C}_{0} - {C}_{( t )} ) ] + {X}_{0} ,$$

wherein:
X₀ is the hairy root biomass concentration at time t₀, and
Y_{X/i} is the apparent biomass yield coefficient (also referred to as biomass yield) empirically pre-determined by means of the following equation:

$$\\ {Y}_{X / i} = \frac{{X}_{1} - {X}_{0}}{{C}_{0} - {C}_{1}}$$

wherein X₁ is the hairy root biomass concentration at a predetermined time t₁;
C₁ is the concentration of the at least one compound at a predetermined time t₁;
X₀ is the hairy root biomass concentration at time t₀; C₀ is the concentration of the at least one compound at time t₀; and i is S for sugar(s) or N for nitrogen; and
e) modifying or adapting or adjusting the conditions of the multi-phasic culture of hairy roots.

The present invention further relates to a method for controlling or maintaining the quality of a multi-phasic culture of hairy roots in a culture medium comprising at least one source of carbon being at least one sugar, and at least one source of nitrogen, the method comprising steps a) to e) as described above, thereby controlling or maintaining the quality of a multi-phasic culture of hairy roots, in particular by correcting an anomaly with the multi-phasic hairy root culture detected by estimating the hairy root biomass concentration X₍ₜ₎ at step d).

In some embodiments, step e) of modifying or adapting or adjusting the conditions of the multi-phasic culture of hairy roots aims at ensuring that the biomass growth rate (μ) as defined herein is optimal. Thus, in some embodiments, step e) of modifying or adapting or adjusting the conditions of the multi-phasic culture of hairy roots aims at ensuring that the hairy root biomass concentration obtained at the end of the multi-phasic culture of hairy roots is optimal.

In some embodiments, the hairy root biomass concentration X₍ₜ₎ estimated at step d) indicates that the biomass growth rate (μ) is suboptimal. As indicated above, comparison of the hairy root biomass concentration X₍ₜ₎ to a reference value may indicate that the biomass growth rate (μ) is suboptimal.

In some embodiments, step e) of modifying or adapting or adjusting the conditions of the multi-phasic culture of hairy roots comprise renewing the culture medium, or modifying the composition of the culture medium (for example by modifying the concentration of one of its constituents, such as one or more sugar(s), a source of sulfate, a source of phosphate, a source of nitrogen, and/or a source of potassium; or by adding a new agent). For example, the composition of the culture medium may be adjusted by modifying the concentration of the at least one source of carbon being at least one sugar, and/or of the at least one source of nitrogen. In particular, the composition of the culture medium may be adjusted by increasing the concentration of the at least one source of carbon being at least one sugar, and/or of the at least one source of nitrogen.

The present invention also relates to a method for maintaining or ensuring the quality of a multi-phasic culture of hairy roots in a culture medium comprising at least one source of carbon being at least one sugar, and at least one source of nitrogen, the method comprising:
a) measuring, at time t₀, the concentration C₀ in the culture medium of at least one compound selected from the group comprising or consisting of at least one sugar and at least one nitrogen source;
b) measuring, at a time t of any one of the culture phases, the concentration C₍ₜ₎ in the culture medium of the at least one compound;
c) calculating the differential concentration C₀-C₍ₜ₎ of the at least one compound;
d) estimating, preferably estimating in real-time, the hairy root biomass concentration X₍ₜ₎ at the time t of any one of the culture phases by means of the following equation:

$$\\ {X}_{( t )} = [ {Y}_{X / i} \times ( {C}_{0} - {C}_{( t )} ) ] + {X}_{0} ,$$

wherein:
X₀ is the hairy root biomass concentration at time t₀, and
Y_{X/i} is the apparent biomass yield coefficient (also referred to as biomass yield) empirically pre-determined by means of the following equation:

$$\\ {Y}_{X / i} = \frac{{X}_{1} - {X}_{0}}{{C}_{0} - {C}_{1}}$$

wherein X₁ is the hairy root biomass concentration at a predetermined time t₁;
C₁ is the concentration of the at least one compound at a predetermined time t₁;
X₀ is the hairy root biomass concentration at time t₀; C₀ is the concentration of the at least one compound at time t₀; and i is S for sugar(s) or N for nitrogen; and
e) stopping the multi-phasic hairy root culture if an anomaly with the multi-phasic hairy root culture is detected by estimating the hairy root biomass concentration X₍ₜ₎ at step d) or continuing the multi-phasic hairy root culture if an anomaly with the multi-phasic hairy root culture is not detected by estimating the hairy root biomass concentration X₍ₜ₎ at step d);
   thereby maintaining or ensuring the quality of a multi-phasic culture of hairy roots.

It is understood that step a) and step b) in the methods described herein comprise measuring (or determining or assessing or detecting), at time to and time t, respectively, the concentrations C₀ and Cₜ in the culture medium of at least one compound selected from the group comprising or consisting of at least one sugar and at least one nitrogen source. In practice, measuring (or determining or assessing or detecting) the concentration of least one sugar and/or at least one nitrogen source is performed by the means of any suitable method acknowledged from the state of the art, or a method adapted therefrom. Illustratively, measuring the concentration of at least one sugar and/or at least one nitrogen source may be performed by mass spectrometry, colorimetry or chromatography, in particular ionic chromatography. It is understood that the concentrations C₀ and Cₜ in the culture medium of least one sugar and/or at least one nitrogen source may be derived from the concentrations of least one sugar and/or at least one nitrogen source measured (or determined or assessed or detected) in a sample of culture medium collected at time t₀ and time t, respectively. In practice, the concentrations are expressed as weight per volume (w/v), in particular as g·L⁻¹.

It is understood that step c) is intended to provide the calculated differential concentration C₀-C₍ₜ₎ of the at least one compound. In other words, the differential concentration C₀-C₍ₜ₎ of the at least one compound represents the consumed concentration of the at least one compound during the time course t of the culture. In practice, the differential concentration C₀-C₍ₜ₎ is expressed as weight per volume (w/v), in particular as g·L⁻¹.

It is understood that step d) is intended to provide an estimation of the hairy root biomass concentration X₍ₜ₎ at the time t. For this purpose, the following equation is used:

$$\\ {X}_{( t )} = [ {Y}_{X / i} \times ( {C}_{0} - {C}_{( t )} ) ] + {X}_{0} .$$

As used herein, X₀ is the hairy root biomass concentration at time to. In practice, X₀ may alternatively represent the dry or fresh biomass concentration of the hairy roots at time to.

In certain embodiments, X₀, as fresh biomass concentration, is measured by collecting a sample of the hairy root culture, removing the water or liquid contained in the sample (originating from the culture medium), weighing the fresh biomass, and calculating the following ratio: weight of the fresh biomass over the initial volume of the sample.

In some embodiments, X₀, as dry biomass concentration, is measured by collecting a sample of the hairy roots culture, removing the water or liquid contained in the sample (originating from the culture medium), drying or lyophilizing the hairy roots, weighing the dry biomass, and calculating the following ratio: weight of the dry biomass over the initial volume of the sample. In certain embodiments, the drying is performed at a temperature of from about 60° C. to about 95° C., during about 16 h to about 72 h. In certain embodiments, the sample is dried at a temperature of about 70° C., during about 48 h.

Within the scope of the invention, the expression “about 60° C. to about 95° C.” encompasses about 60° C., 65° C., 70° C., 75° C., 80° C., 85° C., 90° C. and 95° C. Within the scope of the invention, the expression “about 16 h to about 72 h” encompasses about 16 h, 18 h, 20 h, 22 h, 24 h, 26 h, 28 h, 30 h, 32 h, 34 h, 36 h, 38 h, 40 h, 42 h, 44 h, 46 h, 48 h, 50 h, 52 h, 54 h, 56 h, 58 h, 60 h, 62 h, 64 h, 66 h, 68, 70 h and 72 h.

In certain embodiments, the hairy roots from the collected samples are washed with water, in particular ultrapure water. In practice, the biomass concentration is expressed in dry or fresh weight per volume (w/v), in particular as g_{DW}·L⁻¹ (for dry biomass concentration) or as g_{FW}·L⁻¹ (for fresh biomass concentration).

In certain embodiments, lyophilization of the hairy roots is performed according to any suitable method acknowledged in the state of the art, or a method adapted therefrom. In practice, lyophilization may be performed at a temperature of about −80° C., under vacuum. As used herein, lyophilization may also be referred to as “freeze-drying”.

As used herein, Y_{X/i} is the apparent biomass yield coefficient (also referred to as biomass yield), which is empirically pre-determined by means of the following equation:

$$\\ {Y}_{X / i} = \frac{{X}_{1} - {X}_{0}}{{C}_{0} - {C}_{1}}$$

It is to be understood that the apparent biomass yield coefficient Y_{x/i} is empirically pre-determined. As used herein, the term “empirically pre-determined” is intended to mean that the yield is calculated upon collection of experimental data, instead of being determined by a theory.

As used herein, X₁-X₀ represents the hairy root biomass concentration produced within the time course t₁-t₀ of a culture of hairy roots. In some embodiments, X₀ and X₁ are measured upon collecting a sample of the hairy roots culture at to and a sample of the hairy roots culture at t₁. In certain embodiments, the hairy roots from the collected samples are washed with water, in particular ultrapure water. In practice, the produced hairy root biomass concentration is expressed as dry weight per volume (w/v), in particular as g_{DW}·L⁻¹ or as fresh weight per volume (w/v), in particular as g_{FW}·L⁻¹.

As used herein, C₀-C₁ represents the consumed concentration of the at least one compound (i.e., the at least one compound selected from the group comprising or consisting of at least one sugar and at least one nitrogen source) within the time course t₁-t₀ of a culture of hairy roots. In practice, the consumed concentration of the at least one compound is expressed as weight per volume (w/v), in particular as g_{N}·L⁻¹ when the at least one compound is a nitrogen source, in particular total nitrogen, or as g_{S}·L⁻¹ when the at least one compound is a sugar, in particular total sugars.

In certain embodiments, t₁ is at least about 1 day, preferably at least about 2 days, more preferably at least about 5 days of the culture of hairy roots, i.e., since the start of the culture. In some embodiments, t₁ is comprised from about 5 days to about 20 days, in particular from about 10 days to about 15 days of the culture of hairy roots, i.e., since the start of the culture. Within the scope of the invention, the expression “at least about 1 day” encompasses, 1, 2, 3, 4, 5, 6, 7, 8, 9, 10, 11, 12, 13, 14, 15, 16, 17, 18, 19, 20 day(s) or more.

Without wishing to be bound to a theory, the inventors found that the biomass yield Y_{x/i} surprisingly correlates linearly with the consumed concentration of nitrogen and/or sugar over the entire time course of a multi-phasic culture of hairy roots, and does not correlate linearly with other parameters, such as, e.g., the conductivity of the culture medium, the consumed concentration of other nutrients, such as potassium or sulfate. As used herein, the term “correlation” is intended to mean that the relationship between the biomass yield and the consumed concentration of nitrogen and/or sugar over the entire time course of a culture of hairy roots is statistically significant.

In some embodiments, statistical significance may be achieved with a correlation coefficient (r) above (i.e., greater than) at least about 0.90, preferably above at least about 0.95, more preferably above at least about 0.99. In certain embodiments, statistical significance may be achieved with a regression correlation (r²) above (i.e., greater than) at least about 0.90, preferably above at least about 0.96, more preferably above at least about 0.97. Within the scope of the invention, the expression “at least about 0.90” encompasses 0.90, 0.91, 0.92, 0.93, 0.94, 0.95, 0.96, 0.97, 0.98, 0.99 and 1.

In some embodiments, the at least one sugar is selected from the group comprising or consisting of glucose, fructose, sucrose, and combinations thereof, and preferably comprises or consists of a combination of glucose, fructose and sucrose.

In some embodiments, as used herein, the combination of glucose, fructose and sucrose may be referred to as “total sugars”. In some embodiments, “total sugars” include all the sugars present in the culture medium. In some embodiments, “total sugars” include glucose, fructose, sucrose and any other sugar that may be present in the culture medium, such as, for example, mannose, xylose, galactose and/or ribose.

In certain embodiments, the at least one sugar is total sugars. In some embodiments, total sugars concentration (i.e., the concentration of total sugars) may be measured by the phenol/sulfuric method as disclosed by Dubois et al. (Analytical Chemistry 1956 28 (3), 350-356).

In certain embodiments, when the at least one compound is at least one sugar, the biomass yield Y_{X/S} is comprised between about 0.25 and about 2.50, or ranges from about 0.25 to about 2.50.

As used herein, the expression “between about 0.25 and about 2.50” encompasses about 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30; 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2,40, 2,45 and 2.50. Thus, as used herein, 0.25 and 2.50 are included in the expression “between about 0.25 and about 2.50”.

In some embodiments, the at least one nitrogen source is total nitrogen. As used herein, “total nitrogen” comprises or consists of all the compounds being a source of nitrogen, such as, e.g., ammonium, nitrate, nitrite, amino acids, proteins, and the like. In some embodiments, total nitrogen comprises or consists of ammonium (NH₄⁺) and nitrate (NO₃⁻).

In some embodiments, when the at least one compound is a nitrogen source, the biomass yield Y_{X/N} is comprised between about 5 and about 40, or ranges from about 5 to about 40.

As used herein, the expression “between about 5 and about 40” encompasses about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40. Thus, as used herein, 5 and 40 are included in the expression “between about 5 and about 40”.

In certain embodiments, each phase of the multi-phasic culture is characterized by its own biomass growth rate.

In some embodiments, the biomass growth rate (μ) is calculated with the following equation:

$$\\ μ = \frac{Ln X ( t ) - Ln {X}_{0}}{t}$$

wherein X₍ₜ₎ represents the hairy root biomass concentration at the time t, X₀ represents the hairy root concentration at the time to and t is the time.

In certain embodiments, the biomass growth rate (μ) is expressed as time⁻¹, in particular as h⁻¹ (hour⁻¹) or d⁻¹ (day⁻¹).

According to some embodiments, the multi-phasic culture is a bi-phasic culture.

In certain embodiments, the multi-phasic culture, in particular the bi-phasic culture, comprises:
a) a first phase of culturing hairy roots dedicated to biomass growth; and
b) one or more further phase(s) of culturing hairy roots dedicated to the production of one or more molecule(s) of interest.
   Thus, in some embodiments, “first phase” with regards to a multi-phasic hairy root culture refers to a phase of a biomass growth; and “second phase” with regards to a multi-phasic hairy root culture refers to a phase of production of one or more molecule(s) of interest. In some embodiments, the second phase dedicated to the production of one or more molecule(s) of interest can comprise several cycles of production of one or more molecule(s) of interest. In some embodiments, the second phase dedicated to the production of one or more molecule(s) of interest can be repeated several times. In some embodiments, the multi-phasic hairy root culture can thus comprise several successive second phase dedicated to the production of one or more molecule(s) of interest.

In certain embodiments, the bi-phasic culture comprises:
a) a first phase of culturing hairy roots dedicated to biomass growth; and
b) a second phase of culturing hairy roots dedicated to the production of one or more molecule(s) of interest, which may comprise one or several cycle(s) of production of one or more molecule(s) of interest.

In some embodiments, the first phase and the one or more further phase(s) of culturing hairy roots are performed in one or more culture medium, preferably in one culture medium. In certain embodiments, the first phase and the one or more further phase(s) of culturing hairy roots are performed in the same culture medium. In certain embodiments, the first phase and the one or more further phase(s) of culturing hairy roots are performed in the same culture medium which is renewed at least once between the first phase and any one, or every one, of the one or more further phase(s). In certain embodiments, the first phase and the one or more further phase(s) of culturing hairy roots are performed in distinct culture media. In some embodiments, the multi-phasic culture of hairy roots is performed in a continuous process, preferably in alternance of first and further phases, or alternatively in alternance of one single first phase and multiple further phases. In some embodiments, the multi-phasic culture of hairy roots is performed in a continuous process, preferably in alternance of first and second phases, or alternatively in alternance of one single first phase and multiple second phases.

For each phase of the culturing of the hairy roots, the culture medium may be renewed one or more times, preferably by an identical culture medium, preferably by an identical volume.

As used herein, the term “culture medium” is referring to a solid or liquid medium, preferably a liquid medium, containing nutrients in which hairy roots can be maintained and/or grown. Culture media thus contain all the elements that the hairy roots need for maintenance and/or growth. A suitable culture medium may comprise a carbon source, water, salts, a source of amino acids and a source of nitrogen.

In practice, a suitable culture medium according to the invention may comprise (i) one or more pH buffering system(s); (ii) one or more inorganic salt(s); (iii) one or more trace element(s) (also referred to as microelement(s)), including iron, copper, cobalt, magnesium, and the like; (iv) one or more free amino acid(s); (v) one or more vitamin(s); (vi) one or more hormone(s); (vii) one or more carbon/energy source(s); (ix) one or more macro-elements source(s), including a source of nitrogen, a source of phosphorus, a source of sulfur, and the like.

Culture media for culturing hairy roots are well-known in the art. In some embodiments, a suitable culture medium for culturing hairy roots may be Standard Gamborg's (B5) medium, Murashige and Skoog's (MS) basal medium or N6 medium, which may be commercially acquired. In certain embodiments, the suitable culture medium is a dedicated culture medium specifically developed, which may also be referred to as a “proprietary culture medium” or “home-made culture medium”.

Methods for obtaining hairy roots are well-known to the skilled artisan. As indicated above, hairy roots are root emergences which appear after the infection of a plant or plantlet, notably after a bacterial infection by a Rhizobium rhizogenes bacterium or by a Rhizobium radiobacter bacterium harboring rol genes for example.

In practice, germination and seedling growth of plantlets from which hairy roots will be obtained may occur at a temperature ranging from about 15° C. to about 26° C., preferably from about 20° C. to about 24° C. and more preferably at about 22° C. Within the scope of the instant invention, the expression “about 15° C. to about 26° C.” encompasses about 15° C., 16° C., 17° C., 18° C., 19° C., 20° C., 21° C., 22° C., 23° C., 24° C., 25° C. and 26° C.

In some embodiments, germination and seedling growth may occur in a light/dark photoperiod from about 13 h to about 18 h, preferably from about 15 h to about 17 h, and more preferably at about 16 h. Within the scope of the present invention, the expression “about 13 h to about 18 h” encompasses about 13 h, 14 h, 15 h, 16 h, 17 h and 18 h.

In some embodiments, generated plantlets are infected by a suitable bacterial or viral strain, preferably a bacterial strain of Rhizobium rhizogenes (formerly known as Agrobacterium rhizogenes), or strain of Agrobacterium Tumefaciens harboring rol genes. This leads to the formation of hairy roots.

In some embodiments, the first phase and/or the one or more further phase(s) of culturing hairy roots is/are performed for at least about 1, 2, 3, 4, 5, 6, 7, 8, 9, 10, 11, 12, 13, 14, 15, 16, 17, 18, 19, 20 days, or more. It is understood that the duration of the first phase and/or the one or more further phase(s) of culture may depend on the size of the vessel in which the culture of hairy roots is performed, as larger vessels may necessitate longer duration of the first phase and/or the one or more further phase(s) of culture.

In some embodiments, the multi-phasic culture of hairy roots, including at least a first phase and one or more further phase(s), is performed for at least about 2, 3, 4, 5, 6, 7, 8, 9, 10, 11, 12, 13, 14, 15, 16, 17, 18, 19, 20 days, or more. In some embodiments, the multi-phasic culture of hairy roots, including at least a first phase and one or more further phase(s), is performed for about 2 days to about 100 days. Within the scope of the invention, the expression “for about 2 days to about 100 days” encompasses about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100 days.

In some embodiments, the multi-phasic culture of hairy roots is performed in a culture medium, wherein the culture medium is Gamborg B5 medium further comprising at least one sugar. In some embodiments, the at least one sugar is selected in the group comprising or consisting of sucrose, glucose, fructose, mannose, xylose and ribose. In certain embodiments, the sugar is incorporated in the Gamborg B5 medium at a concentration of from about 0.1% (0.1 g/100 ml; 1 g·L⁻¹) to about 15% (15 g/100 ml; 150 g·L⁻¹), preferably from about 1% to about 5%, more preferably of about 3%. Within the scope of the invention, the expression “from about 0.1% to about 15%” encompasses about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5% and 15%.

In some embodiments, the culture medium may be Gamborg B5 medium with from about 1% to about 5% sucrose (1-5 g/100 ml; 10-50 g·L⁻¹), preferably about 3% sucrose (30 g·L⁻¹). Within the scope of the invention, the expression “from about 1% to about 5%” encompasses about 1%, 1.5%, 2%, 2.5%, 3%, 3.5%, 4%, 4.5% and 5%.

In practice, the one or more further phase(s) of culturing the hairy roots (in particular the phase of production of the one or more molecule(s) of interest by the hairy roots) may be performed in the presence of a chemical and/or biological inductor, or alternatively in the presence of a physical inductor. As used herein, the term “inductor” is intended to refer to a chemical and/or biological entity, or alternatively a physical process, that is employed to initiate the production of the one or more molecule(s) of interest by the hairy roots, within the one or more further phase(s) of the culture, which thus correspond(s) to one or more phase(s) of production of a molecule of interest.

In some embodiments, the one or more further phase(s) of culturing the hairy roots is/are performed in the presence of a chemical and/or biological inductor of the production of the one or more molecule(s) of interest.

In certain embodiments, the chemical and/or biological inductor of the production of the one or more molecule(s) of interest is selected in the group, in particular in the non-exhaustive group comprising or consisting of plant hormones, such as, e.g., auxins, and the like; phytopharmaceuticals, such as, e.g., methoxyfenozide, tubefenozide and the like; steroids, such as, e.g., dexamethasone, estradiol, and the like; alcohols, such as, e.g., ethanol, methanol, and the like; metal ions, such as, e.g., copper, silver, cadmium, cobalt, and the like; antibiotics such as, e.g., tetracycline; polyosides, such as, e.g., cyclodextrins, chitosan, chitin, sucrose, sorbitol, dextran, and the like; polypeptides, such as, e.g., elicitin; inorganic salts, such as, e.g., sodium orthovanadate, vanadyl sulphate, sodium chloride, and the like; organic salts, such as, e.g., methyl jasmonate, jasmonic acid, gibberellic acid, salicylic acid, sodium salicylate, abscisic acid and the like; proline; organic molecule, such as, e.g., polyethylene glycol, tween, PVP (polyvinylpyrrolidone), urea, and the like; yeast extracts; microorganisms, such as, e.g., Trichoderma atroviride, Protomyces gravidus, Claviceps purpurea, Mucor hivernalis, Fusarium oxysporum, Phoma exigua, Botrytis cinerea, Aspergillus niger, Saccharomyces cerevisiae, Agrobacterium rhizogenes, Bacillus subtilis, Bacillus cereus, Escherichia coli, Rhizobium leguminosarum.

In some embodiments, the one or more further phase(s) of culturing the hairy roots is/are performed in the presence of a physical inductor of the production of the one or more molecule(s) of interest.

In certain embodiments, the physical inductor of the production of the one or more molecule(s) of interest is selected in the group, in particular in the non-exhaustive group, comprising or consisting of red light, green light, blue light, temperature, oxygen, pH, ozone, UV-C, osmotic stress, the like and combinations thereof.

In practice, the chemical and/or biological inductor of the production of the one or more molecule(s) of interest may be an agent promoting the induction of rhizocals.

In some embodiments, the one or more further phase(s) of culturing is/are performed in the presence of an agent promoting the induction of rhizocals, also referred to as an inductor of rhizocals. As used herein, the term “rhizocal” or “rhizocallus” refers to a conic-shaped structure connected to the hairy roots, also termed lateral root emergence, which develops alongside of the hairy roots in a solidarized way.

In certain embodiments, for the induction of rhizocals, an inductor of rhizocals is added to the culture medium in the second or further phase of culturing the hairy roots, preferably after about 5 days to about 55 days of the first phase or preceding phase of culturing hairy roots (i.e., after about 5 days to about 55 days since the start of the first phase or preceding phase of the multi-phasic hairy root culture) and preferably after about 14 days of the first phase or preceding phase of culturing hairy roots (i.e., after about 14 days since the start of the first phase or preceding phase of the multi-phasic hairy root culture). Within the scope of the invention, the expression “after about 5 days to about 55 days of culture” encompasses after about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 and 55 days of culture.

In some embodiments, the induction of rhizocals, within the one or more further phase(s) of culturing hairy roots, is performed for about 5 days to about 30 days, preferably for about 10 days to about 25 days, more preferably for about 14 days or about 25 days. In some embodiments, the phase of induction of rhizocals is performed for at least 5 days. Within the scope of the invention, the expression “about 10 days to about 30 days” encompasses after about 10, 11, 12, 13, 14, 15, 16, 17, 18, 19, 20, 21, 22, 23, 24, 25, 26, 27, 28, 29 and 30 days.

In some embodiments, the inductor of rhizocals is a hormone, preferably an auxin.

Within the scope of the invention “an inductor of rhizocals” means that the addition of said inductor of rhizocals in the culture medium leads to the appearance of rhizocals (or rhizocalli) which are lateral root emergences appearing on hairy roots. Hairy roots with rhizocals are able to produce the molecule of interest in a higher quantity than hairy roots with no rhizocals.

In some embodiments, the auxin may be selected from the group comprising or consisting of 2,4-dichlorophenoxyacetic acid (2,4-D), 3-indoleacetic acid (IAA), indole-3-butyric acid (IBA), 1-naphthaleneacetic acid (NAA), 2,4,5-trichlorophenoxyacetic acid (2,4,5 T), 2,3,5-triiodoacetic acid, 4-chlorophenoxyacetic acid, 2-naphthoxyacetic acid, 1-naphthylacetic acid, 4-amino-3,5,6-trichloropicolinic acid, 3,6-dichloro-2-methoxybenzoic acid (Dicamba), derivatives thereof and the like. In some embodiments, the auxin is 2,4-dichlorophenoxyacetic acid (2,4-D).

In some embodiments, during the one or more further phase(s) of culturing, the culture medium thus comprises an auxin, in particular 2,4-D, in a concentration of from about 0.1 mg/L to about 10 mg/L. Within the scope of the invention, the expression “about 0.1 mg/L to about 10 mg/L” encompasses about 0.1, 0.2, 0.3, 0.4, 0.5, 0.6, 0.7, 0.8, 0.9, 1.0, 1.2, 1.4, 1.6, 1.8, 2.0, 2.2, 2,4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8 and 10.0 mg/L.

In some embodiments, during the one or more further phase(s) of culturing, the culture medium is Gamborg B5 medium with about 3% sucrose and 1 mg/L of 2,4-D. The addition of auxin, in particular 2,4-D, allows the formation of rhizocals, leading to an increase of the ability of the roots to synthesize and optionally secrete the molecule(s) of interest. In some embodiments, the addition of auxin may further promote the cessation of the biomass growth.

In practice, the biomass growth rate (μ1) of the first phase of hairy root culture and the biomass growth rate(s) (μ2) of the one or more further phase(s) of hairy root culture may alternatively be distinct or identical.

In some embodiments, the biomass growth rate (μ1) of the first phase of hairy root culture and the biomass growth rate(s) (μ2) of the one or more further phase(s) of hairy root culture are distinct. As used herein, the expression “distinct” is intended to mean that both biomass growth rates do not have the same value. In said embodiments, the biomass growth rate of the first phase of culture is at least 1.2-fold higher than the growth rate(s) of the one or more further phase(s) of culture. Within the scope of the invention, the expression “at least 1.2-fold” encompasses at least 1.2, 1.3, 1.4, 1.5, 1.6, 1.7, 1.8, 1.9, 2.0, 2.2, 2,4, 2.6, 2.8, 3.0, 3.5, 4.0, 4.5, 5.0, 6.0, 7.0, 8.0, 9.0, 10, 15, 20, 25, 30, 35, 40, 45, 50, 60, 70, 80, 90, 100, 200, 300, 400, 500, 600, 700, 800, 900, 1,000-fold, or more.

In some embodiments, the biomass growth rate (μ1) of the first phase of hairy root culture and the biomass growth rate(s) (μ2) of the one or more further phase(s) of hairy root culture are identical or similar. As used herein, the expression “identical or similar” is intended to mean that both biomass growth rates have substantially the same value. In said embodiments, the biomass growth rate of the first phase of culture is strictly less than (i.e., strictly below) 1.2-fold higher or strictly less than 1.2-fold lower than the biomass growth rate(s) of the one or more further phase(s) of culture. Within the scope of the invention, the expression “strictly less than 1.2-fold” (or “strictly below 1.2-fold”) encompasses 1.1, 1.0, 0.9, 0.8, 0.7, 0.6, 0.5, 0.4, 0.3, 0.2, 0.1, 0.05, 0.01, 0.005, 0.001-fold, or less.

In certain embodiments, the biomass growth rate (μ1) of the first phase of hairy root culture is comprised or ranges from about 0.05 to about 1.00. Within the scope of the invention, the expression “about 0.05 to about 1” encompasses about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 and 1.00.

In some embodiments, the multi-phasic culture of hairy roots is characterized by the fact that the biomass growth rate (μ1) of the first phase of hairy root culture and the biomass growth rate(s) (μ2) of the one or more further phase(s) of hairy root culture are substantially identical. In some embodiments, the expression “substantially identical” is intended to mean that the biomass growth rates have values with a difference that is below the 10% statistical margin of error.

In some embodiments, the multi-phasic culture of hairy roots is aimed at producing fresh biomass and/or one or more molecule(s) of interest.

In certain embodiments, the multi-phasic culture of hairy roots is aimed at producing fresh biomass. In some embodiments, the multi-phasic culture of hairy roots is aimed at producing one or more molecule(s) of interest. In certain embodiments, the multi-phasic culture of hairy roots is aimed at producing fresh biomass and one or more molecule(s) of interest.

In some embodiments, the methods as described herein comprising estimating, preferably estimating in real-time, the hairy root biomass concentration X₍ₜ₎ of a multi-phasic culture of hairy roots can also be used for estimating and/or monitoring the amount or quantity of one or more molecule(s) of interest produced by the hairy roots. Indeed, in some embodiments, the amount or quantity of one or more molecule(s) of interest produced by the hairy roots in the multi-phasic hairy root culture is correlated to the hairy root biomass concentration X₍ₜ₎. Thus, in some embodiments, estimating and/or monitoring the hairy root biomass concentration X₍ₜ₎ of a multi-phasic culture of hairy roots allows to estimate and/or monitor the amount or quantity of one or more molecule(s) of interest produced by the hairy roots in the multi-phasic hairy root culture.

In certain embodiments, the one or more molecule(s) of interest is/are selected from the group comprising or consisting of recombinant proteins, metabolites, non-peptidic hormones, structured associations of recombinant proteins, virus-like particles and viruses.

In some embodiments, the recombinant protein is selected from the group comprising or consisting of allergens; vaccines; viral proteins; enzymes; enzyme inhibitors; antibodies; antibody fragments; antigens, toxins; anti-microbial peptides; peptidic hormones; growth factors; blood proteins, in particular albumin, coagulation factors, transferrin; receptors and/or signaling proteins; protein components of biomedical standards; protein components of cell culture media; fusion and/or tagged proteins; cysteine (disulfide bridges)-rich peptides and proteins; and plant proteins, in particular lectins, papain.

In some embodiments, the protein of interest (i.e., the recombinant protein) according to the invention is not naturally produced by the hairy root-based system. In some embodiments, the protein of interest is a protein from an animal species, preferably a mammalian species such as a primate, a canine, a feline, a rodent or an equine species. In some embodiments, the protein of interest is a human protein.

In some embodiments, the protein of interest (i.e., the recombinant protein) according to the invention may be a glycosylated protein. As used herein, the term “glycosylated protein” refers to the result of the enzymatic process that attaches glycans to proteins. Glycosylation is a post-translational modification and glycans play a structural and functional role in membrane and secreted proteins.

As well-known in the art, the most common glycosylation processes are the N-glycosylation and the O-glycosylation. The N-glycosylation refers to the addition of an oligosaccharide harboring a N-acetyl-glucosamine on an asparagine (Asn) amino acid included in the following sequence of a protein: Asn-Xaa-Ser or Asn-Xaa-Thr, with Xaa being any amino acid except proline (Pro), serine (Ser) or threonine (Thr). The O-glycosylation refers to the addition of glycans to an —OH residue of some Ser and Thr amino acids of proteins.

In certain embodiments, the one or more molecule(s) of interest is a metabolite or a non-peptide hormone (also referred herein as non-peptidic hormone). As used herein, “metabolite” refers to a small molecule, i.e., an intermediate or final product of a metabolic reaction.

In some embodiments, the metabolite is a specialized/secondary metabolite of industrial interest. In some embodiments, the metabolite is selected from the group comprising or consisting of polyphenols; alkaloids; cannabinoids; terpenoids and steroids; flavonoids; and tannins, naturally or artificially expressed in plants.

As used herein, “polyphenols, alkaloids, cannabinoids, terpenoids, steroids, flavonoids, and tannins” are intended to refer to their commonly accepted definition in the field, and also encompass derivatives of these compounds. In practice, the term “derivatives” refers to compounds that share similar structures to their counterpart and have similar functions.

In certain embodiments, the one or more molecule(s) of interest is/are secreted in the culture medium. As used herein, the term “secreted”, when referred to a molecule of interest, is intended to mean that the molecule of interest, upon synthesis in the cytoplasm of the cells of the hairy roots, may cross the cellular membrane/envelop of the cells and is to be localized outside these cells, in particular within the culture medium.

In some embodiments, the one or more molecule(s) of interest may be quantitatively and/or qualitatively assessed by any appropriate analytical technique acknowledged from the state of the art, or a method adapted therefrom. Non-limitative examples of suitable analytical techniques include mass spectrometry, gas or liquid chromatography, enzyme-linked immunosorbent assay (ELISA), Western blotting, enzymatic assays, colorimetry, fluorimetry, and the like.

In practice, the hairy roots may alternatively be transgenic hairy roots or non-transgenic hairy roots.

In some embodiments, the hairy roots are transgenic hairy roots. Thus, in some embodiments, the hairy roots are genetically modified. As used herein, the term “transgenic” is intended to refer to hairy roots that comprise one or more heterogeneous nucleic acid sequence(s) encoding the one or more molecule(s) of interest, or the machinery to produce the one or more molecule(s) of interest, in particular one or more nucleic acid sequence(s) that is/are comprised in one or more expression system(s), more particularly one or more vector(s) containing one or more expression cassette(s) comprising one or more gene(s) encoding the one or more molecule(s) of interest.

Within the scope of the invention, the term “expression cassette” refers to a nucleic acid construct which can be introduced in a cell and which allows the expression of the gene comprised in the expression cassette. In practice, a suitable expression cassette may comprise a promotor, a nucleic acid encoding the molecule of interest, a terminator, a signal peptide and optionally regulatory sequences that allow controlling the steps of transcription (e.g., polyA sequence) and/or translation.

In some embodiments, the promotor may be a viral promotor, in particular a viral promotor derived from a Brassicaceae plant-infecting virus. In some embodiments, the promotor may be an inducible promotor, i.e., chemical or physical inducible system (e.g., copper, steroid, alcohol, light), such as, for example, Tet repressor-based, tetracycline de-repressible; tTA-based, tetracycline inactivable; glucocorticoid receptor based, dexamethasone inducible; A1cR-based, ethanol inducible; Ecdysone receptor (EcR)-based, EcR agonist inducible; and estrogen receptor-based, β-estradiol inducible. In practice, a suitable promotor may be a constitutive Cauliflower Mosaic Virus (CaMV) 35S simple or double promotor or the Nos (Nopaline synthase) promotor.

In some embodiments, the expression cassette may comprise one or more regulatory sequence(s). In some embodiments the regulatory sequence may be selected from the group comprising, or consisting of, a TMV Ω enhancer, consensus sequence, and transcriptional factor. In certain embodiments, the regulatory sequence may be a TMV Ω enhancer.

In some embodiments, the expression cassette may comprise a polyadenylation signal that consists of multiple adenosine monophosphates. In practice, the expression cassette may also comprise a CaMV polyA sequence.

In some embodiments, the terminator sequence may comprise a sequence from Agrobacterium tumefaciens (i.e., T-nos, tmas, tocs, tORF25, ttml, tg7), from Solanum tuberosum (i.e., tpinII), from Pisum sativum (i.e., tE9) or from Glycine max (i.e., t₇S). In certain embodiments, a suitable terminator sequence may be a CaMV T35S terminator.

In some embodiments, the expression cassette may comprise a nucleic acid sequence encoding a signal peptide. Within the scope of the invention and as well-known from the state of the art, a “signal peptide” is a short peptide sequence which is, in most of the cases, present at the N-terminus part of a protein and necessary for the protein to cross the cell plasma membrane and therefore be secreted outside the cell.

In certain embodiments, the signal peptide may be the native signal peptide of the protein of interest. In another embodiment, said signal peptide may be derived from a Brassicaceae plant. In practice, a suitable signal peptide may be an Arabidopsis pectin methyl esterase (PME) signal peptide, such as Arabidopsis At1g69940.

In some embodiments, the expression cassette is then cloned into an expression vector, such as, e.g., a plasmid. Typically, the expression vector may be a binary vector suitable for expression in a plant cell, such as the pRD400, pBIN19, pBINPlus or pCAMBIA binary vector. In practice, pBIN19, pBINPlus and pCAMBIA binary vectors may be commercially available from Addgene®. In some embodiments, the plasmid may be a pRD400 plasmid.

Within the invention, the expression vector, e.g., the plasmid, may be incorporated into a competent bacterium by any one of the different processes known from the state of the art, such as bacterial transformation or electroporation.

As used herein, the term “competent” refers to a bacterium that has an increased ability to uptake an extra genomic nucleic acid into its cytoplasm. The skilled artisan is familiar with techniques for preparing competent bacteria (see, e.g., J. Sambrook and D. Russell, Molecular Cloning: A Laboratory Manual, 3^{rd} ed., Cold Spring Harbor Laboratory Press, Cold Spring Harbor, N. Y. (2001)). In some embodiments, competent bacteria for bacterial transformation may be chemically competent cells, in particular calcium chloride treated bacteria.

As used herein, the term “electroporation” refers to a method that consists in the utilization of an electrical field in a solution comprising plasmids and bacteria in order to increase the permeability of the bacteria cell membrane allowing plasmids to be introduced in the bacteria. Suitable protocols may be found, e.g., in Green and Sambrook (Molecular Cloning, 4th Edition, 2012, Cold Spring Harbor Laboratory Press).

Typically, infection of the hairy roots by a bacterium is performed by contacting the bacterium with the hairy roots which has been wounded beforehand, as previously described in the state of the art.

In some embodiments, the bacterium may be Rhizobium rhizogenes (formerly known as Agrobacterium rhizogenes), Rhizobium radiobacter (formerly known as Agrobacterium tumefaciens), or Rhizobium vitis (formerly known as Agrobacterium vitis). In practice, the bacterium used to infect the roots may be Rhizobium rhizogenes (formerly known as Agrobacterium rhizogenes). Many strains of Rhizobium rhizogenes (formerly known as Agrobacterium rhizogenes) can be used. Suitable strains include but are not limited to strain TR7 (or ATCC® 25818 or LBA 9402), A4T, A4, ATCC® 11325, LMG 155, LBA 1334 and ATCC® 15834. In some embodiments, the strain of Rhizobium rhizogenes may be strain TR7 or strain ATCC® 15834, preferably strain ATCC® 15834. In practice, the bacterium may be Rhizobium radiobacter (formerly known as Agrobacterium tumefaciens) harboring the rol genes, genetically integrated. Suitable strains include but are not limited to strain C58, C58C1, LBA4404, GV2260, GV3100, A136, GV3101, GV3850, EHA101, EHA105 and AGL−1. In some embodiments, the strain of Rhizobium radiobacter may be strain GV3101 or strain AGL−1, preferably strain GV3101.

The “rol genes” refer to the group of bacterial genes capable of inducing the formation of roots and also able to affect growth and morphogenetic potential of plant cells, at least in part by altering the capability to respond to plant hormones.

In some embodiments, the hairy roots represent a hairy root-based expression system, in particular a system wherein hairy roots are genetically modified and are used to produce one or more molecule(s) of interest.

In particular embodiments, a molecular construction has been evaluated for its ability to produce the recombinant protein of interest in high yield. This molecular construction is characterized by the use of a 35S double promoter, a TMV Ω enhancer, a PME signal peptide, the nucleic acid encoding the recombinant protein and a 35S terminator.

In some embodiments, the whole sequence is codon-usage optimized for Brassica species taking into account a GC contents around 50-60%. The sequence is then gene-synthesized and cloned into, first, an intermediary plasmid (pUC plasmid), then into the binary plasmid pRD400. The sequencing of the pRD400 plasmid having integrated the molecular construction makes it possible to validate the integrity of the gene construct. The binary plasmid is then incorporated into competent R. rhizogenes bacteria by electroporation. Finally, the incorporation of the plasmid into the transformed R. rhizogenes clone is validated by DNA sequencing. Plantlets of Brassica species are then infected with this recombinant R. rhizogenes clone. The resulting clones are individualized and are all cultured first in solid culture medium, then in liquid culture medium. Antibiotics are only used for the 5 first cycles of culture and are only dedicated to eliminate R. rhizogenes. Apart from this very first step, all the process is antibiotic-free.

The first selection of the genetically modified hairy root clones may be based on their growth capacity. RNA may be extracted from some of these hairy root clones and the integration of the gene encoding the protein of interest is confirmed by RT-PCR.

To refine and simplify the screening of the clones, a specific activity test may be usually set up, as far as it is relevant (e.g., production of enzymes). This test may then be applied on the clones to screen, as well as on samples generated during the different downstream steps. Using this activity assay, it is possible to identify the best producing clone(s).

In alternative embodiments, the hairy roots are non-transgenic hairy roots. Thus, in alternative embodiments, the hairy roots are not genetically modified.

In some embodiments, the hairy roots-based expression system is used to produce an endogenous molecule of interest such as a metabolite or a non-peptidic hormone as described above. As used herein, “endogenous molecule” refers to a molecule which originates from the hairy roots, that is to say which is naturally expressed by the hairy roots, without any requirement for said hairy roots to be genetically modified. For example, hairy roots may thus be used for the production of specialized/secondary metabolites of industrial interest. In some embodiments, the hairy roots used to produce an endogenous molecule of interest are not genetically modified. In some embodiments, the hairy roots used to produce an endogenous molecule of interest are genetically modified.

In some embodiments, the production of an endogenous molecule of interest is artificially induced in the hairy roots-based expression system, by culturing the hairy roots under conditions enabling the production of said molecular compound of interest for example during one or more phase(s) of phase of production of molecules of interest. Conditions enabling the production of endogenous molecules of interest in hairy roots are well-known in the art. Examples of conditions enabling the production of endogenous compounds of interest in roots, in particular in hairy roots, include adding a chemical and/or physical and/or biological inductor to the culture medium, as indicated above.

In some embodiments, the method is performed in a non-sterile environment.

In some alternative embodiments, the method is performed in a sterile environment. In practice, the vessels and/or the culture media may be sterilized according to the protocols known from the state of the art. Non-limitating examples of sterilization treatments include heat-treatment (steam sterilization, high-temperature dry sterilization), UV treatment, and gamma ray treatment.

In some embodiments, the hairy root belongs to a family selected from the group of families comprising or consisting of the Brassicaceae family; the Solanaceae family; the Cannabaceae family; the Caryophyllaceae family; Saponaria family; and the Vitaceae family.

In some embodiments, the hairy root is originating from a species selected in the group of species comprising or consisting of Brassica rapa rapa, Brassica napus, Salvia Milthiorrhiza, Panax Ginseng, Armoracia rusticana, Trigonella foenumgraceum, Lippia dulcis, Lithospermum erythrorhizon, Ophiorrhiza pumila, and Echinacea purpurea, Echinacea Angustifolia, Puerariaphaseoloides, Harpagophytum Procumbens, Morinda Citrifolia, Hypericum Perforatum, Derris trifolia, Salvia miltiorrhiza, Salvia prevalzkii, Echinacea pallida, Cistanche tubulosa, Glycyrrhiza glabra, Sophora flavescens, Rhodiola Rosea, Polygonum cuspidatum, Fallopia multiflora, Lepidium peruvianum, Whitania Somnifera, Astragalus Membranaceous, Berberis Vulgaris, Sanguinaria canadensis, Eleutherococcus Senticosus, Cannabis sativa, Hydrastis Canadensis, Arctium Majus, Piper methysticium, Pueraria lobata, Glycyrrhiza uralensis, Ptychopetalum olacoides, Dioscorea Vollosa, Yucca shidigera, Panax quinquifolium, Azadirachta indica, Catharanthus trichophyllus, Calystegia sepium, Atropa belladonna, Hyoscyamus muticus, Artemisia annua, Datura stramonium, Arabidopsis thaliana, Stizolobium, Hassjoo, Ipomea aquatica, Perilla fruitescnens, Catharanthus roseus, Taxus brevifolia, Gloriosa Superba, Saponaria officinalis, Solanum tuberosum, Nicotiana tabacum, Nicotiana benthamiana, Cannabis sativa, Vitis vinifera, Duboisia leichhardtii, Ducoisia myoporoides and Cinchosa Pubescens.

In certain embodiments, the hairy root is originating from a species selected in the group comprising or consisting of Brassica rapa rapa, Brassica napus and Cannabis sativa.

In certain embodiments, the multi-phasic culture is performed in a bioreactor.

As used herein, the term “bioreactor” refers to a vessel in which a biological process takes place. In practice, the biological process is the growth of a living organism such as hairy roots and/or the production of a compound or molecule of interest.

In some embodiments, the multi-phasic culture is carried out in a volume of culture medium of at least about 20 L, 50 L, 75 L, or 100 L, preferably of at least about 350 L, more preferably of about 500 L.

Within the scope of the invention, the expression “at least about 20 L” encompasses about 20, 25, 30, 35, 40, 45, 50, 60, 70, 80, 90, 100, 120, 140, 160, 180, 200, 220, 240, 250, 260, 280, 300, 350, 400, 450, 500, 600, 700, 800, 900 and 1,000 L, or more.

In certain embodiments, the multi-phasic culture is performed in a bioreactor, preferably in a volume of culture medium of at least about 20 L, 50 L, 75 L, or 100 L, more preferably of at least about 350 L, more preferably of about 500 L.

### BRIEF DESCRIPTION OF THE DRAWINGS

FIGS. 1A-1D are a set of plots showing: (FIG. 1A) the biomass concentration (g_{DW}·L⁻¹) (closed circles) and protein of interest production (relative productivity, in %) (closed squares); (FIG. 1B) the conductivity (mS) (closed squares) and pH (open circles) of the extracellular culture medium; (FIG. 1C) the extracellular sugar concentrations (g_{C}·L⁻¹), including glucose (closed squares), sucrose (closed circles), total sugars IC (sucrose, fructose and glucose concentrations measured by ionic chromatography (IC); crosses), fructose (closed triangles) and total sugars (sucrose, fructose and glucose concentrations; open diamonds); and (FIG. 1D) the evolution of the amounts of the main nutrients (mg·L⁻¹), including sulfate (closed diamonds), ammonium (closed triangles), potassium (crosses), phosphate (closed circles) and nitrate (closed squares); during the growth kinetics of Brassica rapa rapa hairy roots in Erlenmeyer flasks. Dotted vertical line indicates the culture medium renewal and addition of 2,4D.

FIGS. 2A-2C are a set of plots showing the biomass produced in function of the evolution of conductivity decrease (FIG. 2A), the total sugar uptake (FIG. 2B) and the total nitrogen consumption (FIG. 2C). The dotted square represents the growth phase (from day 0 to day 14) and error bars are standard deviations.

FIGS. 3A-3B are a set of histograms showing the biomass measurement and estimations using the correlations given in Table 1 at the end of the growth phase (FIG. 3A) and during the production phase at day 26 (FIG. 3B). The dotted horizontal lines correspond to ±10% of the measured biomass concentration (experimental data). Error bars are standard deviations.

FIGS. 4A-4C are a combination of plots showing the evolution of conductivity (closed squares) and pH (open circles) (FIG. 4A); extracellular sugar concentrations (g_{C}·L⁻¹), including glucose (closed squares), sucrose (closed circles), total sugars IC (sucrose, fructose and glucose concentrations measured by ionic chromatography (IC); crosses), fructose (closed triangles) and total sugars (sucrose, fructose and glucose concentrations; open diamonds) (FIG. 4B); and main nutrient concentrations, including sulfate (closed diamonds), ammonium (closed triangles), potassium (crosses), phosphate (closed circles) and nitrate (closed squares) (FIG. 4C); in the culture medium during the growth phase of Brassica rapa rapa hairy roots in a large-scale bioreactor.

FIG. 5 is a histogram showing the biomass measurement and estimations using correlations determined in small-scale culture conditions (Table 1) at the end of the growth phase of a culture of Brassica rapa rapa hairy roots in a large-scale bioreactor. Dotted horizontal lines represent±10% of the actual biomass concentration measured at the end of the culture. Error bars are standard deviations.

FIGS. 6A-6B are a set of plots showing the biomass concentration (g_{DW}·L⁻¹) over time (in days) during a bi-phasic culture of Brassica napus Westar 8 (FIG. 6A) and Cannabis sativa Santhica 10-70 (FIG. 6B) hairy roots in Erlenmeyer flasks.

FIGS. 7A-7B are a set of plots showing the biomass (g_{DW}·L⁻¹) produced in function of the evolution of conductivity (mS) during a bi-phasic culture of Brassica napus Westar 8 (FIG. 7A) and Cannabis sativa Santhica 10-70 (FIG. 7B) hairy roots in Erlenmeyer flasks.

FIGS. 8A-8B are a set of plots showing the dry weight biomass (g_{DW}·L⁻¹) produced in function of the evolution of the total sugar uptake (g_{S}·L⁻¹) during a bi-phasic culture of Brassica napus Westar 8 (FIG. 8A) and Cannabis sativa Santhica 10-70 (FIG. 8B) hairy roots in Erlenmeyer flasks.

FIGS. 9A-9B are a set of plots showing the dry weight biomass (g_{DW}·L⁻¹) produced in function of the evolution of the nitrogen uptake (g_{N}·L⁻¹) during a bi-phasic culture of Brassica napus Westar 8 (FIG. 9A) and Cannabis sativa Santhica 10-70 (FIG. 9B) hairy roots in Erlenmeyer flasks.

### EXAMPLES

The present invention and disclosure are further illustrated by the following examples.

### Example 1

### Materials and Methods

### 1. Biological Materials

Hairy roots of a transformed Brassica rapa rapa clone producing recombinant α-L-iduronidase (rIDUA; as described in Cardon et al., Plant Biotechnol J. 2019 Feb; 17 (2): 505-516) were grown at 100 rpm and 25° C. in 200 mL of Gamborg's B5 medium, supplemented with 30 g·L⁻¹ of sucrose. For their preservation, hairy roots were sub-cultured each two weeks.

### 2. Hairy Root Culture

The experiments of growth monitoring in shake flasks and in a bioreactor were carried following the same protocol. Culture systems were seeded with 10 g_{FW}·L⁻¹ (gram of fresh weight (FW) per liter) of 11 days old hairy roots of Brassica rapa rapa. After 14 days of growth, roots were transferred into fresh medium with addition of 2,4D (2,4-dichlorophenoxyacetic acid) for the induction of rhizocals which allows to increase the production of recombinant protein, as described in Ekouna et al. (Plant Cell Tiss Organ Cult 131, 601-610 (2017)), and kept for 25 more days.

### 3. Growth Kinetics Experiments in Shake Flasks

The experiments of growth monitoring in shake flasks were made in 250 mL Erlenmeyer flasks filled with 100 mL of Brassica growth medium (BGM) medium (proprietary culture medium, specifically developed for the culture of Brassicaceae hairy root). The flasks were inoculated with 10 g_{FW}·L⁻¹ of 11 days old hairy roots and maintained at 100 rpm and 25° C. To follow the evolution of the culture, hairy roots were harvested in triplicates at regular time intervals. The harvested roots were washed with ultrapure water and dried at 70° C. for 24 hours to measure the dry weight (DW) of hairy roots. The pH and conductivity of the culture medium were also measured using a HI 5221 pH meter (Hanna Instruments®, Woonsocket, RI, US) and samples were taken and stored at −20° C. for further analysis of sugar and nutrient concentrations. Three different experiments at different dates were done for the growth phase (the first 14 days) and only one for the whole culture process (14 days of growth and 25 days in rhizocals). For the presentation of the growth kinetics in Erlenmeyer flasks, means were calculated based on the three growth phase experiments, with the associated standard deviations.

### 4. Characterization in Large Scale Bioreactor

The characterization of the cultures in large scale was carried out in a 25 liters air-lift bioreactor, filled with 25 L of BGM medium. The bioreactor was seeded with 10 g_{FW}·L⁻¹ of 11 days old hairy roots, aerated and mixed by air injection at 2 L·min⁻¹ and maintained in a thermostatically controlled chamber at 25° C. At regular time intervals, samples of culture medium were taken in sterile conditions and stored at −20° C. for further analysis of sugar and nutrient concentrations, and pH (Steamline®, SI Analytics, Weilheim Germany) and conductivity (Sentek® K10S7, Stepney, Australia) were measured. At the end of the culture, the hairy roots were harvested from the bioreactor, washed with ultrapure water and dried at 70° C. during 48 h to measure the dry weight.

### 5. Culture Analysis

### 5.1. Biomass

The biomass concentration X (in gram of dry weight (g_{DW}) per liter or g_{DW}·L⁻¹) was obtained from the dry weight measurement, dividing the mass of dried roots (in g_{DW}) by the total volume of culture (0.1 L and 25 L for cultures in flasks and bioreactor, respectively).

### 5.2. Sugars and Nutrients

The concentrations of anions (nitrate, sulfate and phosphate), cations (ammonium and potassium) and sugars (sucrose, glucose and fructose) in the culture medium were determined by ionic chromatography (Dionex ICS-3000; Thermo Fisher Scientific®). Anions were quantified using an IonPAC AS11-HC (4×250 mm) ion exchange column (Thermo Fisher Scientific®) thermostatically at 30° C., linked to an ASRS 300 (4 mm) suppressor (at 248 mV) and a conductivity detector. The elution gradient was composed of the following steps: 98% of H₂O and 2% of NaOH 100 mM from 0 to 5 min, 94% of H₂O and 6% of NaOH 100 mM from 5 to 30 min, 60% of H₂O and 40% of NaOH 100 mM from 30 to 45 min, 98% of H₂O and 2% of NaOH 100 mM from 45 to 55 min, at a flowrate of 1 mL·min⁻¹. Before injection, samples were filtered on 0.45 μm cellulose membrane. Standard ranges were made with standard anion solutions (CPAchem®). Cations were quantified using an IonPAC CS19 (4×250 mm) ion exchange column (Thermo Fisher Scientific®) thermostatically at 30° C., linked to an CSRS 300 (4 mm) suppressor (at 147 mV) and a conductivity detector. The elution gradient was the same as the anions' one replacing the NaOH solution by a 50 mM methanesulfonic acid solution. Sugars were measured with a ProPac PAI (4×250 mm) ion exchange column (Thermo Fisher Scientific®) and an amperometric detector. The elution gradient was composed of the following steps: 70% of H₂O and 30% of NaOH 100 mM from 0 to 8.4 min, 40% of H₂O and 60% of NaOH 100 mM from 8.4 to 10 min, 100% of NaOH 100 mM from 20 to 30 min, 100% of sodium acetate 1M and NaOH 100 mM from 30 to 35 min, 70% of H₂O and 30% of NaOH 100 mM from 35 to 45 min, at a flowrate of 1 mL·min⁻¹. Before injection, samples were filtered on 0.45 μm cellulose membrane. Standard ranges were made with HPLC grade sucrose, glucose and fructose. Monitoring and peak areas determination were done by the software Chromeleon 7.0. For better comparison, all sugars were expressed in gram of carbon per liter dividing the concentration in g·L⁻¹ by the molar mass of the concerned sugar (M_{Glc}=M_{Fru}=180 g/mol and M_{Suc}=342 g/mol) and multiplying it by the number of carbon atoms and the molar mass of carbon (M_{C}=12 g/mol).

### 5.3. Total Sugars

The concentration of total sugars was determined using the phenol-sulfuric method (Dubois et al., Analytical Chemistry 1956 28 (3), 350-356). For the dosage of total sugars in the culture medium, 0.2 mL of a 5% aqueous phenol solution (Sigma Aldrich®, ref #: P4557) was added to 0.4 mL of carbohydrate sample. 1 mL of 95-98% sulfuric acid (Sigma Aldrich®, ref #: 258105) was then quickly added to the mixture. After 10 minutes at room temperature, samples were vortexed for 15 sec and incubated in a water bath at room temperature for 20 min, in order to stop the reaction. Calibration samples consisted in glucose (Amresco®, ref #: 0188) solutions at 0, 0.02, 0.04, 0.06, 0.08 and 0.1 g/L prepared in ultrapure water. Absorbance of samples was then measured at 490 nm (maximal intensity of the glucose peak) and 750 nm (to measure the turbidity). The concentration of total sugars in gram of carbon per liter (g_{C}·L⁻¹) was then calculated based on the calibration curve.

### 5.4. Estimation of Growth Rate and Apparent Biomass Yields

For the characterization experiments in flasks, the biomass growth was represented as exponential and the growth rate (μ) was estimated from the regression of the exponential growth phase, according the following equation (1):

$$\\ {X}_{( t )} = {X}_{0} {e}^{{}_{} μ t}$$

wherein X₍ₜ₎ represents the time dependent biomass concentration in g_{DW}·L⁻¹, X₀ represents the initial biomass concentration, μ represents the growth rate in d⁻¹ and t represents the time in days.

The apparent biomass yield coefficient Y_{X/i} represents the amount of biomass produced for a quantity of substrate (i.e., at least one compound comprised within the culture medium) degraded over time (e.g., carbon (such as sugars), nitrogen, sulfate, etc.), according to the following equation (2):

$$\\ {Y}_{X / i} = \frac{{X}_{1} - {X}_{0}}{{C}_{0} - {C}_{1}}$$

wherein X₁ is the hairy root biomass concentration at a predetermined time t₁; C₁ is the concentration of the at least one compound (i.e., substrate) at a predetermined time t₁; X₀ is the hairy root biomass concentration at time t₀; C₀ is the concentration of the at least one compound (i.e., substrate) at time t₀; and i is for the at least one compound (i.e., substrate), for example, S for sugar(s) or N for nitrogen.

The biomass yield can be therefore estimated from the linear phase of the evolution of the quantity of biomass produced X₁-X₀ in function of the amount of substrate consumed C₀-C₁. Even if it is not considered as a substrate, the conductivity indirectly represents the concentration of nutrients in the culture medium and therefore a ratio between the biomass produced and the decrease in conductivity can also be calculated on the same basis as the biomass yield presented above.

### 5.5. Measurement of Recombinant Protein Production

The activity of rIDUA was measured with a fluorimetry assay by using the fluorogenic substrate sodium 4-methylumbelliferyl-α-L-Iduronide (4MU-I) (Santa Cruz Biotechnology®, Dallas, US) as described in Ou et al. (Mol Genet Metab. 2014 Feb; 111 (2): 113-5). Before drying, hairy roots were washed in a saline solution to recover the protein of interest. 25 μL of substrate (400 μM 4MU-I prepared in 0.4M Sodium formate, pH 3.5, as assay buffer) were added to 25 μL of protein samples and were incubated at 37° C. during 30 min. 200 μL of glycine carbonate buffer pH 9.8 were added to the mixture to stop the reaction. 4-methylumbelliferone (4MU) (Sigma Aldrich®, Saint-Louis, US) was used to prepare the standard calibration curve. Fluorescence was measured using a plate reader (TECAN Infinite M1000®, Männedorf, Switzerland) with excitation at 355 nm and emission at 460 nm. Enzyme activity was first measured in μmol of product formed per minute and per sample volume. The productivity was then calculated by dividing the enzyme activity by the corresponding biomass concentration and the culture time and was then expressed in % of the maximum productivity.

### Results

1. Growth Characterization of a Transgenic Clone of Brassica rapa Rapa Hairy Roots in Shake Flasks

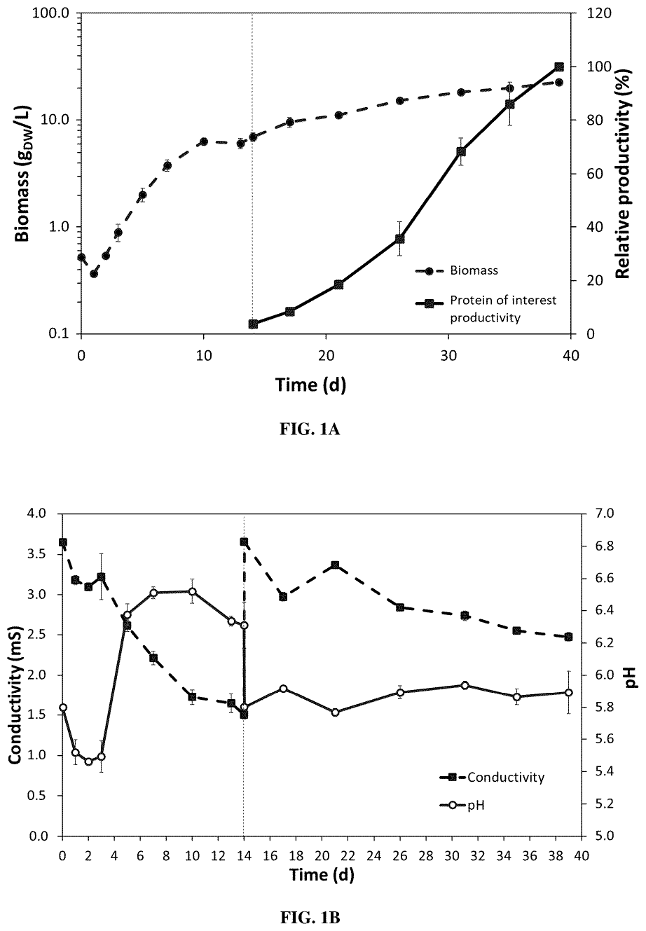

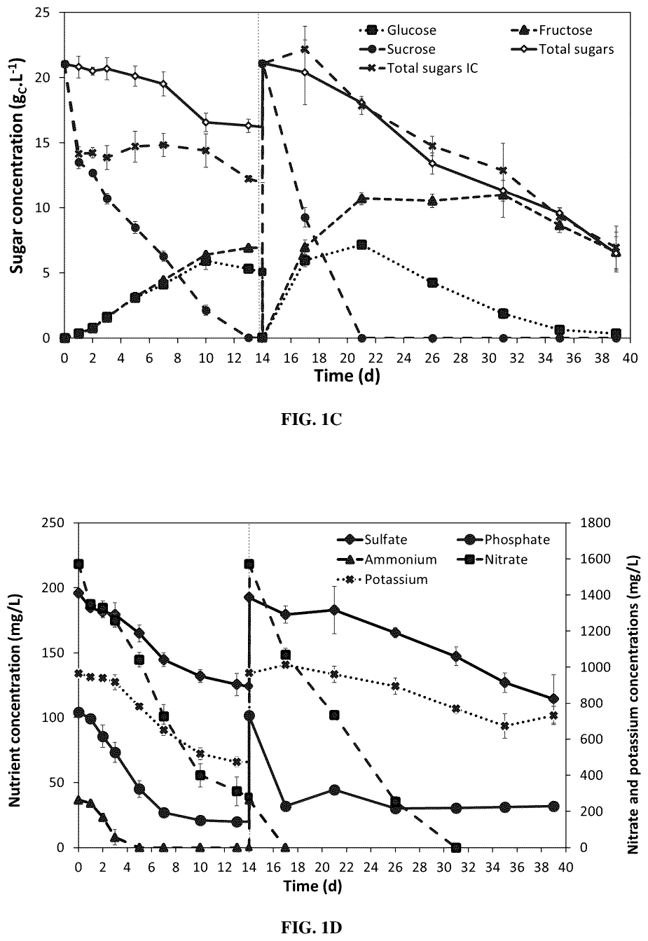

The growth of Brassica rapa rapa hairy roots during 39 days in 100 mL of BGM medium in Erlenmeyer flasks, corresponding to 14 days of biomass growth followed by 25 days of production through rhizocal culture, is presented in FIG. 1A. From day 2 to day 7, the growth of hairy roots was exponential with a maximum growth rate μₘₐₓ=0.34±0.02 d⁻¹, corresponding to a doubling time of 2.04±0.12 d. The biomass concentration reached 6.99±0.63 g_{DW}·L⁻¹ before the culture medium renewal at the 14^{th} day of culture. The standard deviation of U max was less than 10% despite the mean calculated from three different experiments carried out at three different dates, which shows the strong reproducibility and repeatability of the process. After the culture medium renewal at day 14 and the addition of 2,4D for the induction of rhizocals, the hairy roots growth was strongly slowed down, with a maximum growth rate of 0.04±0.01 d⁻¹ (doubling time of 17.33±5.77 d) and reached a final biomass concentration of 22.59±0.09 g_{DW}·L⁻¹. FIG. 1A also shows the relative production of recombinant protein (rIDUA) after the culture medium renewal. A progressive increase of the protein production was observed, which confirms the capacity of the clone to produce recombinant protein in liquid cultures.

The growth of the hairy roots was linked to a decrease of the conductivity of the culture medium, from 3.65 to 1.65 mS during the growth phase and from 3.65 to 2.48 mS during the rhizocal phase, due to the consumption of the ions needed for the biomass growth (FIG. 1B).

The evolution of the different sugars (sucrose, glucose, fructose and total sugars) was followed during the whole culture (FIG. 1C). The concentration of sucrose progressively decreased from the beginning of the culture. The decrease was concomitant with an increase of the concentrations of glucose and fructose in equimolar amounts. This was characteristic of the hydrolysis of sucrose which was complete at day 13. As indicated by the total sugar analysis, the carbon uptake was very low during the seven first days with only 1.54 g_{C}·L⁻¹ consumed (about 7.3% of the initial amount). A more important decrease of the total sugar concentration occurred after day 7 (3.2 g_{C}·L⁻¹), which corresponded to the beginning of glucose uptake. After the culture medium change at day 14, sucrose was completely hydrolyzed in 6 days. Glucose uptake began 3 days after the culture medium renewal and was progressively consumed until the end of the production phase. When the glucose concentration became lower than 2 g_{C}·L⁻¹, fructose started to be consumed from day 31 to the end of the culture. The total sugars uptake was 4.75 g_{C}·L⁻¹ at the end of the growth phase and 14.61 g_{C}·L⁻¹ at the end of the production phase.

The nutrient composition of the culture medium was also analyzed during the culture by following the concentrations of the main cations and of the main anions (FIG. 1D). Ammonium was quickly and fully consumed during the first five days of the culture and 90% of the nitrate amount was consumed during the 14 days of the growth phase. During the same period, an uptake of 81% of the phosphate source was observed and seemed to stabilize after 7 days of growth around 20 mg·L⁻¹. Potassium and sulfate sources were also consumed but to a lesser extent (51% and 36% respectively). After the culture medium renewal and the induction of rhizocals, the nutrient uptake confirmed that nitrogen and phosphate were the main consumed compounds. Indeed, ammonium was completely consumed in only 3 days and the whole initial amount of nitrate was consumed at the 31^{th} day. Phosphate uptake followed the same kinetics during the production phase as during the growth phase, with 66% of the initial phosphate amount consumed until reaching a steady concentration around 30 mg·L⁻¹.

Nutrient monitoring shows that hairy roots of Brassica rapa rapa are able to assimilate both ammonium (NH₄⁺) and nitrate (NO₃⁻) as nitrogen source for amino acids production, as most plant species. In the culture described herein, NH₄⁺ was preferably consumed over NO₃⁻ between day 0 and 3 corresponding to the beginning of the exponential growth phase and a decrease of the culture medium pH. The uptake of phosphate during the growth phase occurred from the beginning of the culture until day 7, which corresponds to the exponential growth phase and the moment where the pH is maximum.

Thus, in the culture conditions described above, nitrogen was the main limiting compound. As the main carbon source, glucose which was completely consumed at the end of the production phase, could also be a potential limitation, even if fructose took over as the secondary carbon source.

### 1.2. Sugars, Nitrogen and Conductivity Correlations for the Estimation of the Biomass Concentration

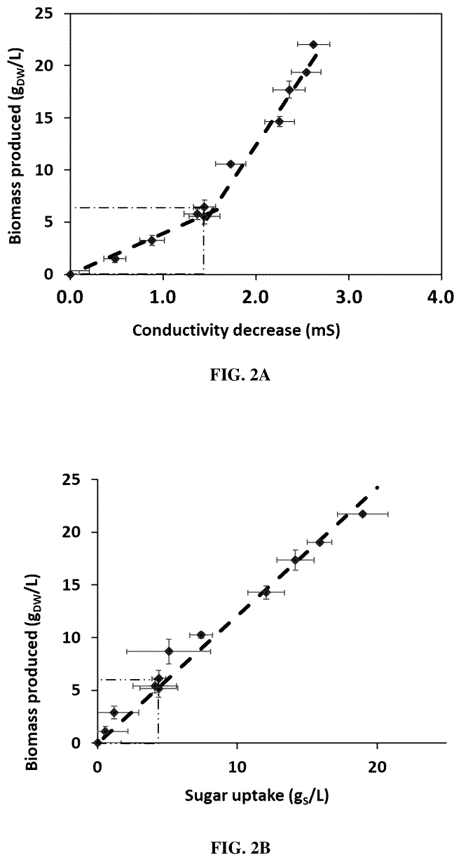

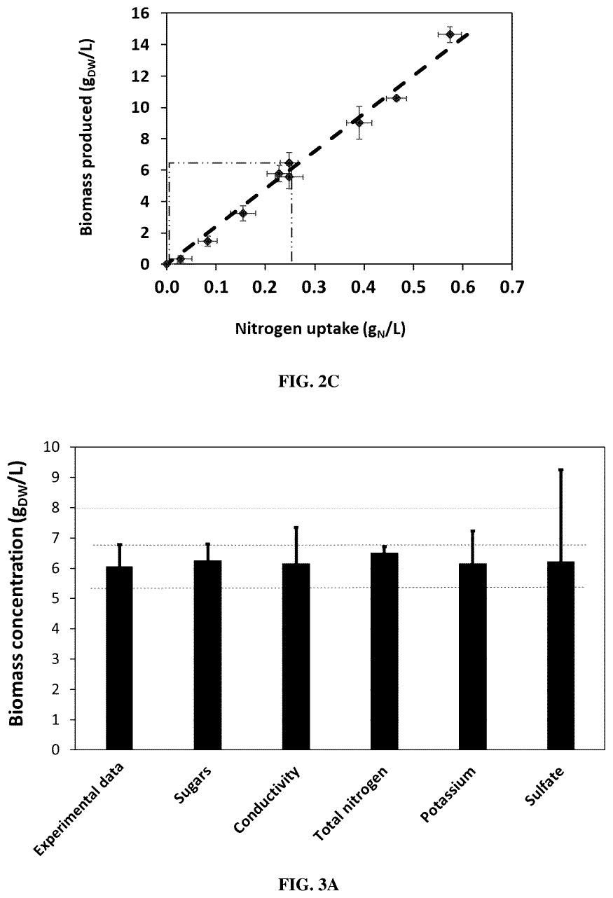

Thanks to the different measurements performed for each sampling point, the biomass concentration evolution was correlated to the uptake of total sugars, total nitrogen, potassium and sulfate, and to the evolution of conductivity, in order to better understand and characterize the culture kinetics of Brassica rapa rapa hairy roots in shake flasks. FIGS. 2A-2C represents the evolution of the biomass produced in function of the conductivity decrease (FIG. 2A), the total sugars uptake (FIG. 2B) and the total nitrogen consumption (FIG. 2C). As expressed with Equation 2, this representation allows to obtain the apparent yield Y_{X/i} of the biomass X in function of a substrate S, provided that the evolution is linear.

Even if the conductivity of the culture medium is not a substrate, strictly speaking, it directly represents the concentration of ions in the culture medium and therefore the nutrient concentration. As presented in FIG. 2A, the conductivity decrease is linearly correlated to the biomass production, both during the growth phase (first phase of the culture of hairy roots) and the rhizocal phase (second phase of the culture of hairy roots), with apparent biomass yields of 3.90±1.11 g_{DW}·L⁻¹·ms⁻¹ and 12.12±3.30 g_{DW}·L⁻¹·ms⁻¹ respectively, regression coefficients r² of 0.965 and 0.970 respectively and a correlation coefficient r=0.985 for both phases. Moreover, taking into account the standard deviations, all the experimental points are fitted with the linear regression.

By representing the evolution of the biomass production in function of the total sugar uptake (i.e., from sulfuric-phenol method and expressed in g_{C}·L⁻¹), a good linear correlation can be highlighted between these two parameters with an apparent biomass yield Y_{X/S}=1.21±0.23 g_{DW}·g_{C}⁻¹, a correlation coefficient r=0.994 and a regression coefficient r²=0.978 (FIG. 2B). Strikingly, the same correlation and apparent biomass yield Y_{X/S} apply during both the growth phase (first phase) and the production phase (second phase), which means that the carbon requirement for the hairy roots is constant during the entire bi-phasic culture. As well as for conductivity, all the experimental data are well fitted with the linear regression, taking into account the standard deviations.

FIG. 2C represents the evolution of biomass production in function of the total nitrogen uptake (addition of ammonium and nitrate uptake, both expressed in gram of nitrogen (g) per liter). It shows that, when nitrogen is not limiting, the biomass production is linearly correlated with the nitrogen uptake with a biomass yield of 24.05±2,44 g_{DW}·g_{N}⁻¹ both during the growth (first phase) and the production (second phase) phases, a correlation coefficient r=0.995 and a regression coefficient r²=0.988. Therefore, the nitrogen requirement for the biomass is constant during the entire bi-phasic culture, as is the case for the sugar requirement.

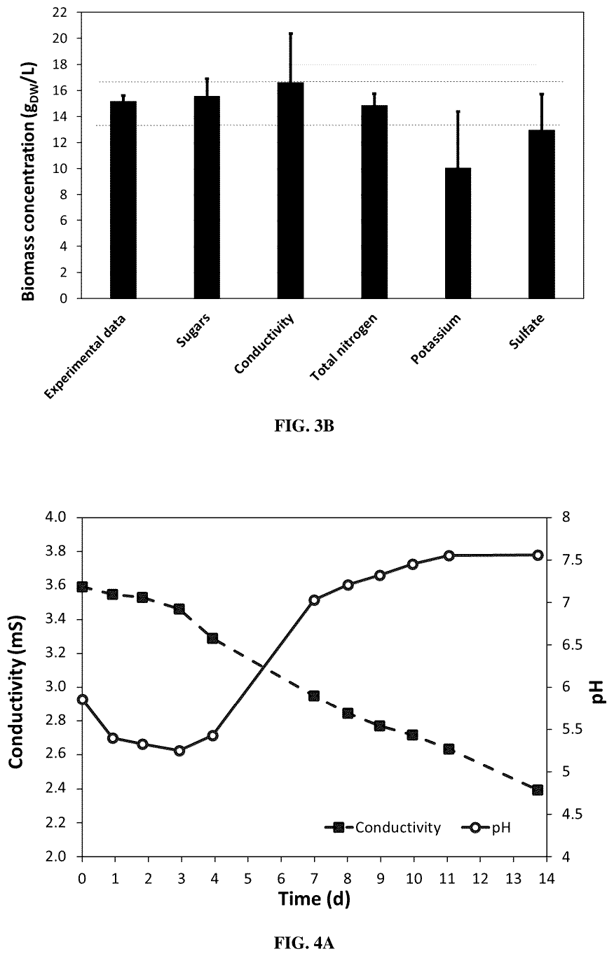

The correlations for the 5 parameters are given in Table 1 below. FIGS. 3A-3B compare the biomass actually measured (experimental data) and the biomass estimations obtained with the 5 correlations, at the end of the growth phase (first phase) (FIG. 3A) and during the production phase (second phase) (FIG. 3B).

TABLE 1
Correlations derived from conductivity (σ), total sugars (S), total
nitrogen (N), potassium (K) and sulfate (Su) measurements during a bi-
phasic culture in shake flasks, for estimation of the biomass concentration
X₍ₜ₎ in function of time t. Subscripts “0” refer to initial time.

| | Apparent biomass yield | |
|---|---|---|
| | Growth phase | Production phase |
| | (first phase) | (second phase) |
| Conductivity (σ) | Y_{X/σ} = 3.90 ± 1.11 (r = 0.985) | Y_{X/σ} = 12.20 ± 3.30 (r = 0.985) |
| Total sugars (S) | Y_{X/S} = 1.21 ± 0.23 (r = 0.994) | |
| Total nitrogen (N) | Y_{X/N} = 24.05 ± 2.44 (r = 0.995) | |
| Potassium (K) | Y_{X/K} = 11.44 ± 1.44 (r = 0.991) | Y_{X/K} = 47.75 ± 14.54 (r = 0.891) |
| Sulfate (Su) | Y_{X/Su} = 99.10 ± 33.33 (r = 0.989) | Y_{X/Su} = 229.97 ± 68.27 (r = 0.975) |

The biomass concentrations estimated at the end of the growth phase (first phase) are all within the ±10% range of the experimental data but only sugars and nitrogen estimations lead to a standard deviation below 10% (FIG. 3A). For the production phase (second phase), all the correlations gave estimated biomass concentrations within the ±10% range of the experimental data, considering the standard deviations of the correlations (FIG. 3B). However, the average value of the estimated biomass from potassium and sulfate correlations are outside the ±10% range of the experimental data, with a large standard deviation which indicates an underestimated prediction.

These correlations are particularly interesting for the estimation of the biomass concentration during a large-scale production where the biomass cannot be measured before the end of the production. So, the on-line and direct measurement of the conductivity of the culture medium with a conductivity probe (Sentek® K10S7 for example) could be a good growth marker to control and estimate the biomass concentration during the culture, using the corresponding apparent biomass yield Y_{X/σ}. However, when performing a multi-phasic culture, comprising for example a growth phase (first phase) and a production phase (second phase), it should be noted that biomass yields Y_{X/σ} must be pre-determined for each phase of the culture. Indeed, as illustrated in FIG. 2A and Table 1, with conductivity, a single biomass yield Y_{X/σ} does not apply in the different phases of a multi-phasic culture.

A regular sampling of culture medium to measure the total nitrogen evolution (easily measured using colorimetric kits (Merck®, ref #114763, for example) or by liquid chromatography) or total sugar evolution (with the phenol-acid method for example) can thus constitute an appropriate and more convenient way to estimate the biomass evolution during a large scale production, in particular for a multi-phasic culture.

1.3. Growth of Hairy Roots of Brassica rapa Rapa in Large Scale Bioreactor

A culture of Brassica rapa rapa hairy roots was carried out in a pilot-scale bioreactor (25 L of useful volume), with the same method as in small scale, i.e., seeded with 10 g_{FW}·L⁻¹ of 11 days old hairy roots, with a growth phase of 14 days.

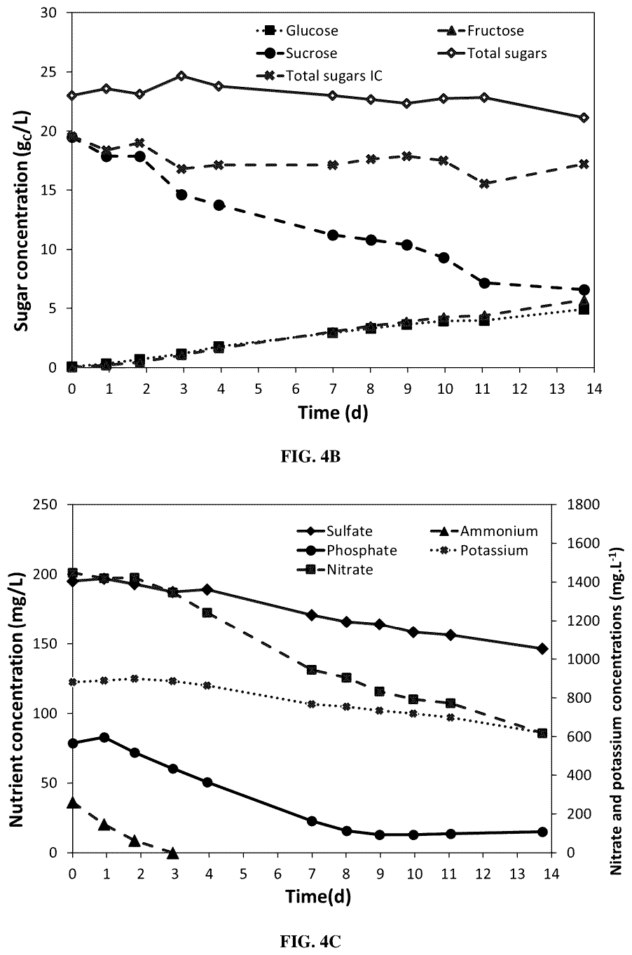

At the end of the growth phase, hairy roots were harvested, dried and weight to measure the biomass produced which was 3.24 g_{DW}·L⁻¹. The Brassica rapa rapa hairy roots culture monitoring is presented in FIGS. 4A-4C, with the evolution of conductivity (FIG. 4A), the evolution of extracellular sugars (FIG. 4B) and the evolution of the main nutrients used for the growth (FIG. 4C). As the biomass concentration cannot be measured during the process, but only after harvest at the end of the culture, only the conductivity and the pH of the culture medium were monitored (FIG. 4A).

During the first 3 days of the culture, a very small decrease of the culture medium conductivity from 3.59 to 3.46 mS was observed, concomitant with a decrease from 5.86 to 5.25 of the pH. An important decrease of the conductivity was then measured from 3.46 to 2.39 mS between day 3 and day 14, concomitant with an increase of the pH from 5.25 to 7.56. Regarding the sugar evolution (FIG. 4B), a progressive decrease in sucrose was observed from the beginning of the culture to the end, mostly due to hydrolysis into glucose and fructose, even if a direct uptake of the sucrose could be possible during the first 3 days, considering the small decrease in total sucrose during this period (from 19.6 to 16.8 g_{C}·L⁻¹, according to the ionic chromatography measurement). The total sugar concentrations remained almost stable from day 3 to day 10, which attests that only hydrolysis of sucrose happened during this period without any extracellular carbon fixation. After the 10^{th} day, a slow-down in glucose accumulation and a small decrease of the total sugar concentration were observed, showing the start of glucose uptake.

FIG. 4C shows the evolution of the main ions during the growth phase in a large-scale bioreactor. Ammonium was completely consumed during the first 3 days and nitrate level was steady until day 2 before it progressively decreased until the end of the growth phase (first phase) from 1,421 to 619 mg·L⁻¹ (42% of the initial nitrate level still remained at the end of the growth phase). An important uptake of phosphate also occurred between day 1 and day 8 with almost 80% of the initial amount consumed, which then stabilized at a basal level around 15 mg·L⁻¹ until the end of the culture. Sulfate and potassium were less consumed with respectively 25% and 29% of initial amounts.

Compared with the cultures in Erlenmeyer flasks (FIG. 1), the culture in large scale bioreactors led to a longer lag time at the beginning of the growth phase (first phase). Indeed, although it was not possible to directly measure the biomass concentration during the process, several markers showed this delay. First, the nutrient consumption was slower to start: this was clearly reflected in the conductivity evolution whose main decrease began on day 3 while it started from the first day in small scale cultures. Nitrate consumption also showed this delay as it only began between day 2 and day 3 versus day 1 for the culture in Erlenmeyer flasks. Second, the hydrolysis of sucrose, which is correlated to invertases and sucrose synthase concentrations and therefore biomass concentration, was much slower than in Erlenmeyer flasks (≈6 g_{C}·L⁻¹ vs 21 g_{C}·L⁻¹ in 14 days).

Despite the delay observed, the growth of Brassica rapa rapa hairy roots in a large-scale bioreactor remained similar to the growth in small scale cultures. Indeed, the decrease of the culture medium conductivity from 3.65 mS to 2.7 mS during the growth phase showed a consumption of the nutrients which was due to the biomass production, and the evolution of pH was similar to that observed in small scale cultures with a decrease from 5.8 to 5.0 at the beginning of the culture followed by an increase to reach a pH of 6.7 at the end of the growth phase (FIG. 4A). However, the biomass concentration reached at the end of the growth phase was lower than in small scale cultures (3.24 g_{DW}·L⁻¹ in large bioreactor and 6.99 g_{DW}·L⁻¹ in Erlenmeyer flasks). This could be caused by the lag time observed in the large bioreactor which would have reduced the time dedicated to the exponential growth phase. The dynamics of sugars was similar to that observed in small scale cultures with hydrolysis of sucrose into fructose and glucose in equimolar amounts from the beginning of the culture, and a consumption of glucose before fructose. Evolution of nutrients was also similar to what was observed in small scale cultures, with a large uptake of nitrogen, first characterized by a quick and total uptake of ammonium and followed by nitrate consumption. Even if nitrogen was the most and main consumed nutrient, uptake of phosphate was also important and reached an equivalent basal concentration to that observed in small scale cultures (around 15-20 mg·L⁻¹) and at the same culture period (between 7 and 8 days of growth).

1.4. Estimation of the Biomass Concentration with the Small-Scale Correlations

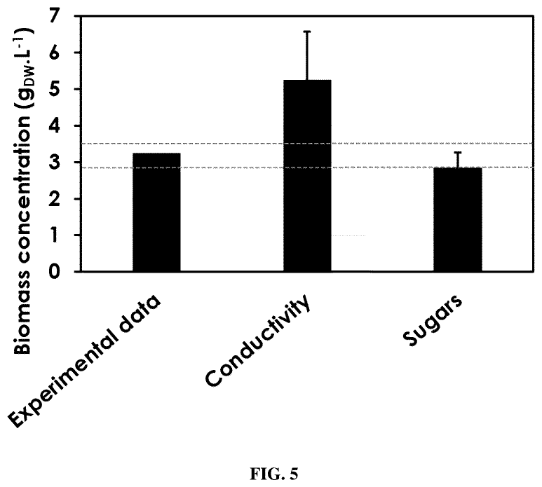

As it is not possible to directly measure the biomass amount inside the large scale bioreactor during the culture, the correlations for the biomass estimation established in small scale cultures (Table 1) were used to see if it is possible to obtain an accurate estimation of the biomass concentration in a large culture (FIG. 5). The actual biomass concentration measured after harvesting the culture was 3.24 g_{DW}·L⁻¹. Considering a ±10% range around the experimental biomass value, the estimations of the biomass concentration at the end of the growth phase are statistically identical when obtained with sugar correlation. The conductivity correlation over-estimated the biomass concentration.

Strikingly, similarly to the observations made in small-scale conditions, it is possible to estimate accurately the biomass concentration (within the 10%-margin of error) in a large-scale culture of hairy roots via the measurement of sugar concentrations in the culture medium.

### Example 2

### Materials and Methods

Brassica rapa rapa hairy roots were grown as in Example 1.

Wild type Brassica napus hairy roots were grown at 100 rpm and 25° C. in 200 mL of Gamborg's B5 medium, supplemented with 30 g·L⁻¹ of sucrose. For their preservation, hairy roots were sub-cultured every two weeks.

Cannabis sativa hairy roots were grown at 70 rpm and 25° C. in 200 mL of MS medium, supplemented with 30 g·L⁻¹ of sucrose. For their preservation, hairy roots were sub-cultured every two weeks.

Culture analysis with respect to biomass, sugars and nutrients uptakes, estimation of growth rate and apparent biomass yields, and measurement of recombinant protein production were performed as in Example 1.

### Results

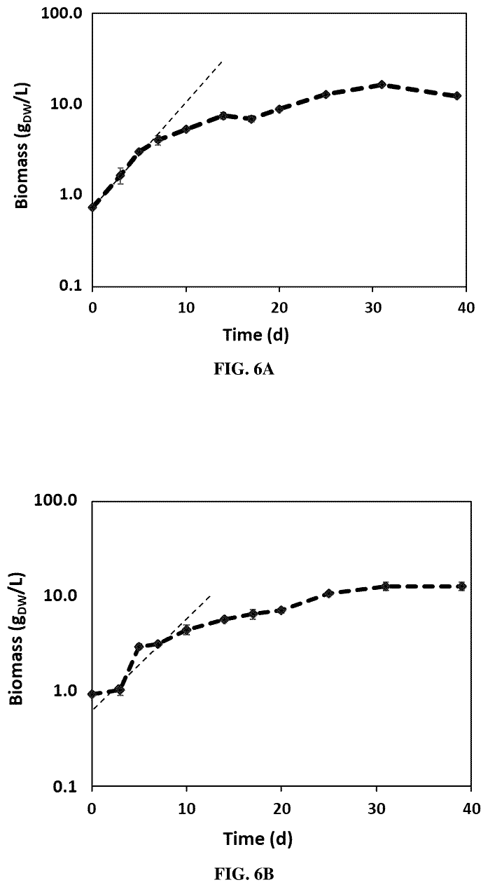

As shown in FIGS. 6A-6B, the growth of Brassica napus and Cannabis sativa hairy roots species during 39 days in 100 mL of BGM medium in Erlenmeyer flasks, consisting of 14 days of biomass growth (first phase) and then 25 days of production through rhizocal culture (second phase), followed the same trend as the growth of Brassica rapa rapa hairy roots in identical conditions (see FIG. 1A for comparison). However, the growth rate was lower for both Brassica napus and Cannabis sativa hairy roots species, as compared to the growth rate of Brassica rapa rapa hairy roots, as seen in Table 2.

TABLE 2
Maximal biomass growth rate and doubling time in
multi-phasic cultures of three Brassica species.
Maximal biomass growth rate μ (d⁻¹)
Doubling time t_{D} (d)

| B. rapa rapa | B. napus | C. sativa |
|---|---|---|
| μ = 0.40 d⁻¹ | μ = 0.26 d⁻¹ | μ = 0.19 d⁻¹ |
| t_{D} = 1.73 d | t_{D} = 2.66 d | t_{D} = 3.65 d |

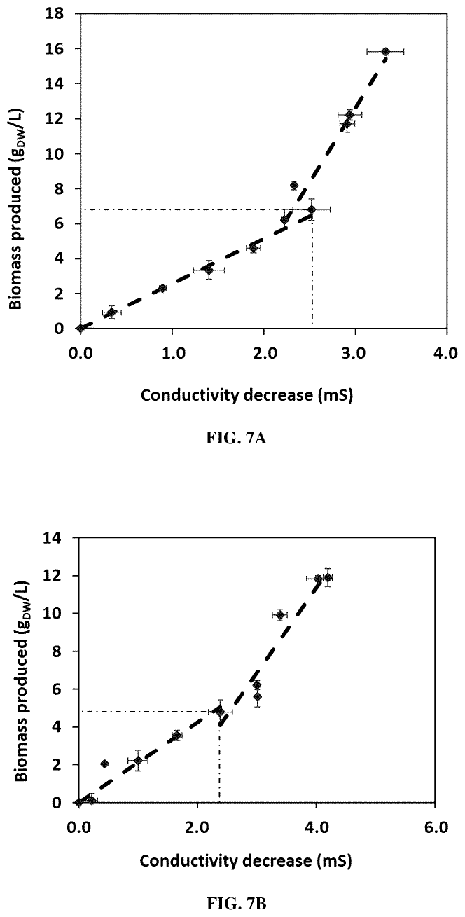

Similarly to Brassica rapa rapa hairy roots (see FIG. 2A), the measured conductivity (mS) for both Brassica napus and Cannabis sativa hairy roots is linearly correlated to the biomass production, both during the growth phase (first phase) and the rhizocal phase (second phase or production phase), with distinct apparent biomass yields for each phase (FIGS. 7A-7B). For Brassica napus hairy roots the biomass yields for conductivity are 2.57±0.48 g_{DW}/L/mS and 8.48±2.58 g_{DW}/L/mS, for the first phase of biomass production and for the second phase of rhizocal induction, respectively (see Table 3); whereas the biomass yields for conductivity during the first phase of biomass production and the second phase of rhizocal induction are respectively 2.11±0.42 g_{DW}/L/mS and 4.49±3.42 g_{DW}/L/mS, for Cannabis sativa hairy roots (see Table 3).

TABLE 3
Biomass growth yield for conductivity in
multi-phasic cultures of three Brassica species.
Biomass growth yield for conductivity (g_{DW}/L/mS)

| | B. rapa rapa | B. napus | C. sativa |
|---|---|---|---|
| Growth phase | 3.90 ± 1.11 | 2.57 ± 0.48 | 2.11 ± 0.42 |
| (first phase) | | | |
| Production phase | 12.20 ± 3.30 | 8.48 ± 2.58 | 4.49 ± 3.42 |
| (second phase) | | | |

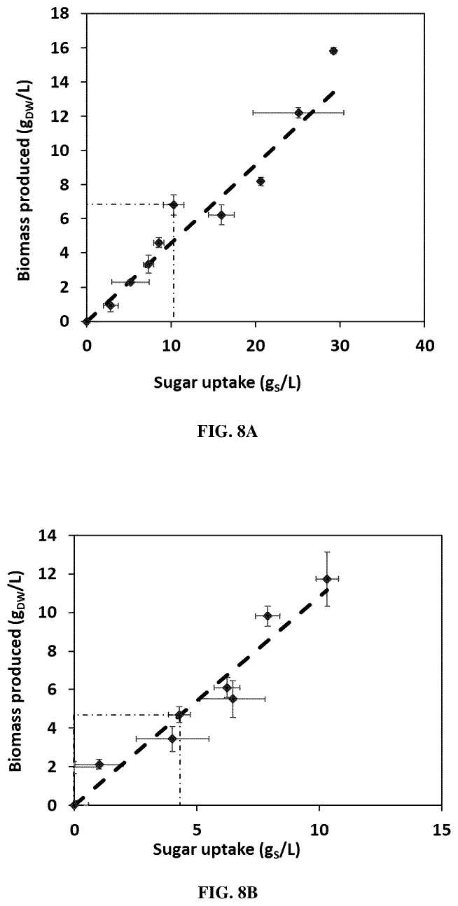

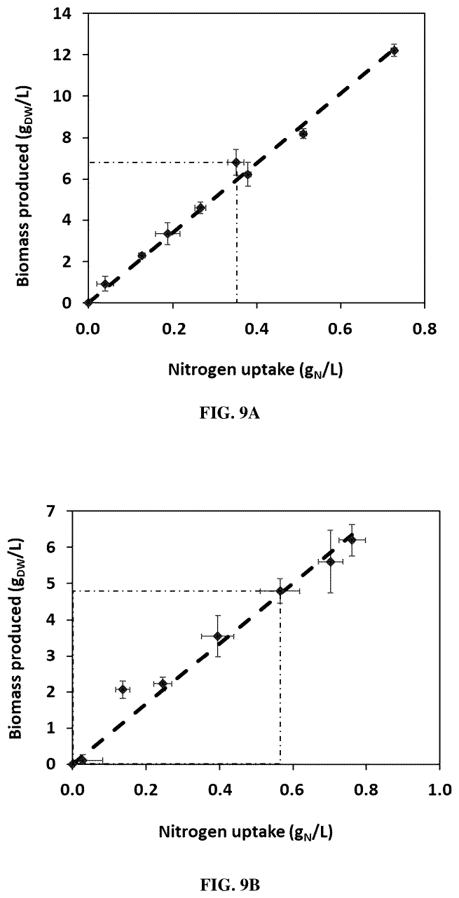

Again, similarly to Brassica rapa rapa, the biomass growth yield for total sugars and for total nitrogen for both Brassica napus and Cannabis sativa hairy roots are identical when growth phase and production phase are compared see FIGS. 8A-8B and Table 4; FIGS. 9A-9B and Table 5; respectively.

TABLE 4
Biomass growth yield for total sugars in
multi-phasic cultures of three Brassica species.
Biomass growth yield for total sugars (g_{DW}/g_{S})

| | B. rapa rapa | B. napus | C. sativa |
|---|---|---|---|
| Growth phase | 1.21 ± 0.23 | 0.49 ± 0.01 | 1.08 ± 0.27 |
| (first phase) | | | |
| Production phase | 1.21 ± 0.23 | 0.49 ± 0.01 | 1.08 ± 0.27 |
| (second phase) | | | |

TABLE 5
Biomass growth yield for total nitrogen in
multi-phasic cultures of three Brassica species.
Biomass growth yield for total nitrogen (g_{DW}/g_{N})

| | B. rapa rapa | B. napus | C. sativa |
|---|---|---|---|
| Growth phase | 24.05 ± 2.44 | 16.91 ± 0.73 | 8.35 ± 1.24 |
| (first phase) | | | |
| Production phase | 24.05 ± 2.44 | 16.91 ± 0.73 | 8.35 ± 1.24 |
| (second phase) | | | |

Consequently, the total sugar and/or total nitrogen consumption provide a useful biomarker to assess the biomass production of hairy roots, at any time of the phase of a multi-phasic culture of the hairy roots in a culture medium, i.e., either at the growth phase (first phase) or the production phase (second phase).

1-15. (canceled)

16. A method for estimating the hairy root biomass concentration X₍ₜ₎ of a multi-phasic culture of hairy roots in a culture medium, the culture medium comprising at least one source of carbon being at least one sugar, and one source of nitrogen, the method comprising:
a) measuring, at time t₀, the concentration C₀ in the culture medium of at least one compound selected from the group consisting of at least one sugar and at least one nitrogen source;
b) measuring, at a time t of any one of the culture phases, the concentration C₍ₜ₎ in the culture medium of the at least one compound;
c) calculating the differential concentration C₀-C₍ₜ₎ of the at least one compound;
d) estimating the hairy root biomass concentration X₍ₜ₎ at the time t of any one of the culture phases by means of the following equation:
$$\\ {X}_{( t )} = [ {Y}_{X / i} \times ( {C}_{0} - {C}_{( t )} ) ] + {X}_{0} ,$$
wherein:
X₀ is the hairy root biomass concentration at time t₀, and
Y_{X/i} is the biomass yield empirically pre-determined by means of the following equation:
$$\\ {Y}_{X / i} = \frac{{X}_{1} - {X}_{0}}{{C}_{0} - {C}_{1}}$$
wherein X₁ is the hairy root biomass concentration at a predetermined time t₁; C₁ is the concentration of the at least one compound at a predetermined time t₁; X₀ is the hairy root biomass concentration at time t₀; C₀ is the concentration of the at least one compound at time t₀; and i is S for sugar(s) or N for nitrogen.

17. The method according to claim 16, wherein the at least one sugar is selected from the group consisting of glucose, fructose, sucrose, and any combinations thereof.

18. The method according to claim 16, wherein the at least one sugar is selected from the group consisting of a combination of glucose, fructose and sucrose.

19. The method according to claim 16, wherein each phase of the multi-phasic culture is characterized by its own biomass growth rate.

20. The method according to claim 16, wherein the multi-phasic culture is a bi-phasic culture.

21. The method according to claim 16, wherein the multi-phasic culture comprises:
a) a first phase of culturing hairy roots dedicated to biomass growth; and
b) one or more further phase(s) of culturing hairy roots dedicated to the production of one or more molecule(s) of interest.

22. The method according to claim 16, wherein the multi-phasic culture of hairy roots is aimed at producing fresh biomass and/or one or more molecule(s) of interest.

23. The method according to claim 16, wherein the one or more molecule(s) of interest is/are selected from the group consisting of recombinant proteins, metabolites, non-peptidic hormones, structured associations of recombinant proteins, virus-like particles and viruses.

24. The method according to claim 23, wherein the recombinant protein is selected from the group consisting of allergens; vaccines; viral proteins; enzymes; enzyme inhibitors; antibodies; antibody fragments; antigens, toxins; anti-microbial peptides; peptidic hormones; growth factors; blood proteins, in particular albumin, coagulation factors, transferrin; receptors and/or signaling proteins; protein components of biomedical standards; protein components of cell culture media; fusion and/or tagged proteins; cysteine (disulfide bridges)-rich peptides and proteins; and plant proteins, in particular lectins, papain.

25. The method according to claim 23, wherein the metabolite is selected from the group consisting of polyphenols; alkaloids; cannabinoids; terpenoids and steroids; flavonoids; and tannins.

26. The method according to claim 16, wherein the hairy root is selected from the group of families consisting of the Brassicaceae family; the Solanaceae family; the Cannabaceae family; the Caryophyllaceae family; the Saponaria family; and the Vitaceae family.

27. The method according to claim 16, wherein the hairy root is selected from the group of species consisting of Brassica rapa rapa, Brassica napus, Salvia Milthiorrhiza, Panax Ginseng, Armoracia rusticana, Trigonella foenumgraceum, Lippia dulcis, Lithospermum erythrorhizon, Ophiorrhiza pumila, and Echinacea purpurea, Echinacea Angustifolia, Puerariaphaseoloides, Harpagophytum Procumbens, Morinda Citrifolia, Hypericum Perforatum, Derris trifolia, Salvia miltiorrhiza, Salvia prevalzkii, Echinacea pallida, Cistanche tubulosa, Glycyrrhiza glabra, Sophora flavescens, Rhodiola Rosea, Polygonum cuspidatum, Fallopia multiflora, Lepidium peruvianum, Whitania Somnifera, Astragalus Membranaceous, Berberis Vulgaris, Sanguinaria canadensis, Eleutherococcus Senticosus, Cannabis sativa, Hydrastis Canadensis, Arctium Majus, Piper methysticium, Pueraria lobata, Glycyrrhiza uralensis, Ptychopetalum olacoides, Dioscorea Vollosa, Yucca shidigera, Panax quinquifolium, Azadirachta indica, Catharanthus trichophyllus, Calystegia sepium, Atropa belladonna, Hyoscyamus muticus, Artemisia annua, Datura stramonium, Arabidopsis thaliana, Stizolobium, Hassjoo, Ipomea aquatica, Perilla fruitescnens, Catharanthus roseus, Taxus brevifolia, Gloriosa Superba, Saponaria officinalis, Solanum tuberosum, Nicotiana tabacum, Nicotiana benthamiana, Cannabis sativa, Vitis vinifera, Duboisia leichhardtii, Ducoisia myoporoides and Cinchosa Pubescens.

28. The method according to claim 21, wherein the one or more further phase(s) of culturing the hairy roots is/are performed in the presence of a chemical and/or physical and/or biological inductor of the production of the one or more molecule(s) of interest.

29. The method according to claim 16, wherein when the at least one compound is at least one sugar, the biomass yield Y_{X/S} ranges from about 0.25 to about 2.50.

30. The method according to claim 16, wherein when the at least one compound is a nitrogen source, the biomass yield Y_{X/N} is ranges from about 5 to about 40.

31. The method according to claim 16, wherein the multi-phasic culture is performed in a bioreactor, in a volume of culture medium of at least about 20 L.

32. The method according to claim 31, wherein the multi-phasic culture is performed in a bioreactor, in a volume of culture medium of at least about 350 L.

33. The method according to claim 31, wherein the multi-phasic culture is performed in a bioreactor, in a volume of culture medium of at least about 500 L.
